(19) United States
(12) Patent Application Publication
(10) Pub. No.: US 2003/0003565 A1
(43) Pub. Date: Jan. 2, 2003

(54) Functional lentiviral vector from an MLV-based backbone

(76) Inventors: Thomas W. Dubensky JR., Piedmont, CA (US); Mehdi Gasmi, San Diego, CA (US); Sybille Sauter, Del Mar, CA (US)

Correspondence Address: CHIRON CORPORATION, Intellectual Property - R440, P.O. Box 8097, Emeryville, CA, 94662-8097, US

(21) Appl. No.: 09/996,073

(22) Filed: Nov. 27, 2001

Related U.S. Application Data
(60) Provisional application No. 60/253,419, filed on Nov. 27, 2000.

(51) Int. Cl. A61K 39/21; C12N 7/00; C12N 15/867

(52) U.S. Cl. 435/235.1; 435/456; 435/320.1; 424/188.1

(57) ABSTRACT

Disclosed are gene therapy vectors based on chimeric murine leukemia virus-feline immunodeficiency virus gene therapy vectors which are suitable for a wide variety of gene therapy applications. Also disclosed are related packaging cell lines, methods for production, and methods of use.

### CROSS-REFERENCE TO RELATED APPLICATIONS

The present invention claims the benefit of provisional application No. 60/253,419, filed Nov. 27, 2000 under the provisions of 35 U.S.C. 119. The disclosure of 60/253,419 is herein incorporated by reference in its entirety.

### TECHNICAL FIELD

The present invention relates generally to pharmaceutical compositions and methods, and more particularly, to chimeric murine leukemia virus-feline immunodeficiency virus gene therapy vectors which are suitable for a wide variety of gene therapy applications.

### BACKGROUND OF THE INVENTION

Since the discovery of nucleic acids in the 1940's and continuing through the most recent era of biotechnology, substantial research has been undertaken in order to realize the possibility that the course of disease may be affected through interaction with the nucleic acids of living organisms. Most recently, a wide variety of methods have been described for altering or affecting genes, including for example, viral vectors derived from retroviruses, adenoviruses, vaccinia viruses, herpes viruses, and adeno-associated viruses (see Jolly, Cancer Gene Therapy 1(1):51-64, 1994).

Of these techniques, recombinant retroviral gene delivery methods have been most extensively utilized, in part due to: (1) the efficient entry of genetic material (the vector genome) into cells; (2) an active, efficient process of entry into the target cell nucleus; (3)relatively high levels of gene expression; (4) the potential to target particular cellular subtypes through control of the vector-target cell binding and the tissue-specific control of gene expression; (5) a general lack of pre-existing host immunity; and (6) substantial knowledge and clinical experience which has been gained with such vectors.

Briefly, retroviruses are diploid positive-strand RNA viruses that replicate through an integrated DNA intermediate. In particular, upon infection by the RNA virus, the retroviral genome is reverse-transcribed into DNA by a virally encoded reverse transcriptase that is carried as a protein in each retrovirus. The viral DNA is then integrated pseudo-randomly into the host cell genome of the infecting cell, forming a “provirus” which is inherited by daughter cells. Moloney MLV is an cotropic virus whose envelope attaches to mouse and rat cells but not to human cells. 4070A MLV is an amphotropic virus whose envelope attaches to cells of mouse and human origin. In the case of Moloney MLV, the precise target for virus attachment is a constrained peptide loop in the third extracellular domain of the murine cationic amino acid transporter (CAT1) which also functions as a Moloney virus receptor when transplanted into the corresponding site on a homologous human protein (Albritton et al, 1993 J Virol 67 p2091-2096).

One major disadvantage of MLV-based vectors, however, is that the host range (i.e., cells infected with the vector) is limited, and the frequency of transduction of non-replicating cells is generally low. Other non-human retroviral vectors, for example vectors derived from feline immunodeficiency virus (FIV), also result in low quantities of genomic vector RNA and low titers of vector producing cell lines (VPLCs). See, e.g., International Publication WO 99/15641, published Apr. 1, 1999.

Thus, there remains a need for compositions and methods that result in high quantities and high frequency of transduction lentiviral vector systems

### SUMMARY OF THE INVENTION

The present invention provides new, chimeric gene therapy delivery vehicles based in-part upon the feline immunodeficiency virus and in part on the MLV. Briefly stated, the present invention provides gene therapy and other nucleic acid delivery vehicles which are based upon a feline immunodeficiency viruses (“FIV”) within an MLV vector backbone. The FIV based vector portion may contain wild type LTRs or hybrid LTRs at one or both ends of the vector. The chimeric vectors can produce, in high titer, FIV-based gene delivery vectors. The invention also provides for related packaging cell lines. Thus, the invention also provides other, related, advantages.

Within one aspect, a chimeric vector of the following general structure is provided: a 5′ MLV LTR, a tRNA binding site, an MLV packaging signal, a 5′ FIV LTR, an internal promoter operably linked to one or more genes of interest, a 3′ FIV LTR, and MLV origin of second strand DNA synthesis, and a 3′ MLV LTR.

Within another aspect of the invention, the FIV vector is nested in a reverse orientation into an MLV vector genome, the entire vector containing a 5′ MLV LTR, a tRNA binding site, an MLV packaging signal, a 3′ FIV LTR, one or more genes of interest operably linked to an internal promoter, an FIV packaging signal, a 5′ FIV LTR, an MLV origin of second strand DNA synthesis and a 3′ MLV LTR.

Within one embodiment the internal promoter is a tissue specific promoter, or alternatively, a promoter such as CMV or SV40. Within further embodiments, the internal FIV vector further comprises an internal ribosome entry site. Within other embodiments, the vector has a nuclear transport element selected from the group consisting of MPMV, HBV, RSV and lentiviral Rev-responsive-elements.

Within yet other embodiments the FIV LTR is composed of less than 25% wild type FIV LTR sequence, and/or FIV LTR contains at least one non-FIV promoter/enhancer. Further, promoter may be operably linked to two genes of interest which are separated by less than 120 nucleotides.

Within various embodiments, the MLV/FIV chimeric vector expresses a gene of interest. Representative examples of suitable genes of interest include selectable markers, cytokines, factor VIII, factor IX, LDL receptor, prodrug activating enzymes, trans-dominant negative viral or cancer-associated proteins, and tyrosine hydroxylase.

Within other aspects of the invention, packaging expression cassettes are provided comprising a promoter and a sequence encoding FIV gag/pol. Within other embodiments, the cassette further comprising an element selected from the group consisting of vif, ORF 2 or rev.

Within another aspect vif expression cassettes are provided comprising a promoter and a sequence comprising at least one of vif rev or ORF 2, wherein the promoter is operably linked to vif, rev or ORF 2. Within a related aspect, amphotropic envelope expression cassettes are provided comprising a promoter and a sequence encoding amphotropic env, wherein the promoter is operably linked to said virus.

Also provided are host cells (e.g., of human, dog, cat or murine origin) which contain an expression cassette as described above.

Within further aspects packaging cell lines are provided comprising an expression cassette comprising a promoter operably linked to a sequence encoding FIV gag/pol (including dUTPage), an expression cassette comprising a promoter operably linked to a sequence encoding an envelope, and a nuclear transport element, wherein said promoter is operably linked to said sequence encoding gag/pol. Within further embodiments, the packaging cell line further comprises a sequence encoding one or more of vif rev or ORF 2. Within preferred embodiments, the expression cassette is stably integrated within the cell, and/or upon introduction of a FIV vector construct, produces particles at a concentration of greater than 10³ cfu/ml. Within preferred embodiments, the promoter is inducible.

Within preferred embodiments of the invention, the packaging cell lines produce particles that are free of replication competent virus. Within further aspects, methods of producing high titer gene delivery vehicles are provided. In certain embodiments, the methods involve using a chimeric MLV-FIV vector construct to generate FIV vector particles carrying a gene of interest. The chimeric vectors are packaged, for instance by transiently transfecting the chimeric vectors into suitable cells (e.g., 293T cells) along with an env-expression cassette (e.g., VSV-G plasmid) and a gag/pol construct (e.g., pSCV10). The resulting particles, which contain the entire MLV-FIV sequence, can be concentrated and use to transfect FIV PCLs at high moi. In suitable FIV PCLs, only the FIV vector RNA is transcribed and only the FIV packaging signal recognized, thereby generating, with high efficiency, vector particles which include the FIV vector sequences without MLV sequences.

These and other aspects of the present invention will become evident upon reference to the following detailed description and attached drawings. In addition, various references are set forth below which describe in more detail certain procedures or compositions (e.g., plasmids, etc.), and are therefore incorporated by reference in their entirety as if each were individually noted for incorporation.

### BRIEF DESCRIPTION OF THE DRAWINGS

FIG. 1 schematically illustrates the genomic organization of MLV/FIV Chimeric construct.

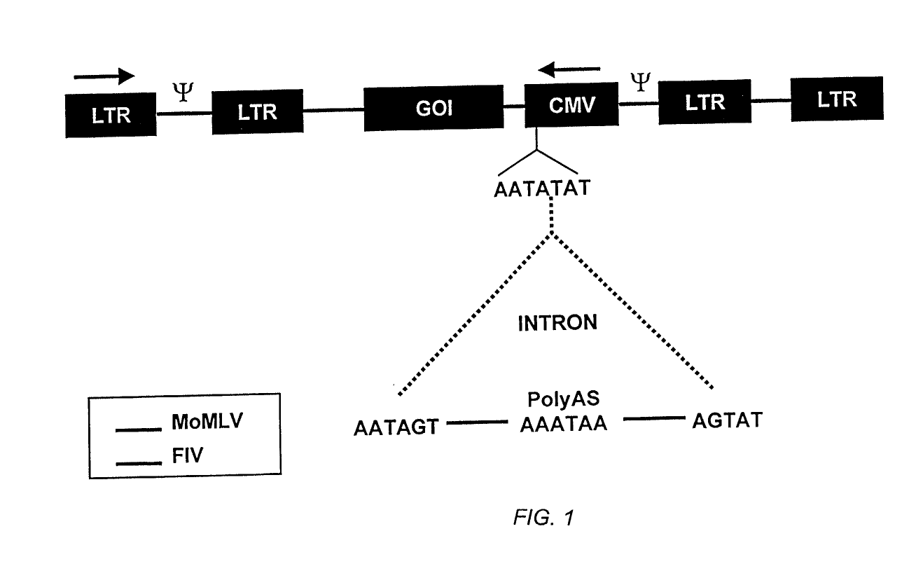

FIG. 2 is a blot depicting expression levels of the 70 Kd amphotropic envelope protein.

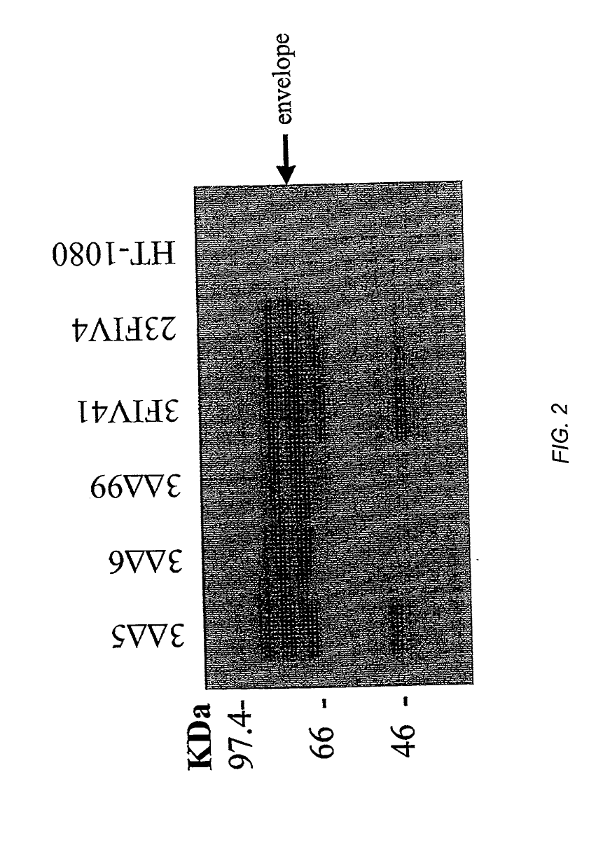

FIG. 3, panels A-F, are FACS analyses detecting the amphotropic envelpe on the cell surface of the 5 FIV PCLs compared to the MLV-based PCL HA-LB. The profile for the negative controls cells (HT-1080) is shown in darker gray.

FIG. 4 is a blot depicting FIV p24 capsid in pelleted supernatant from 5 FIV PCLs and controls.

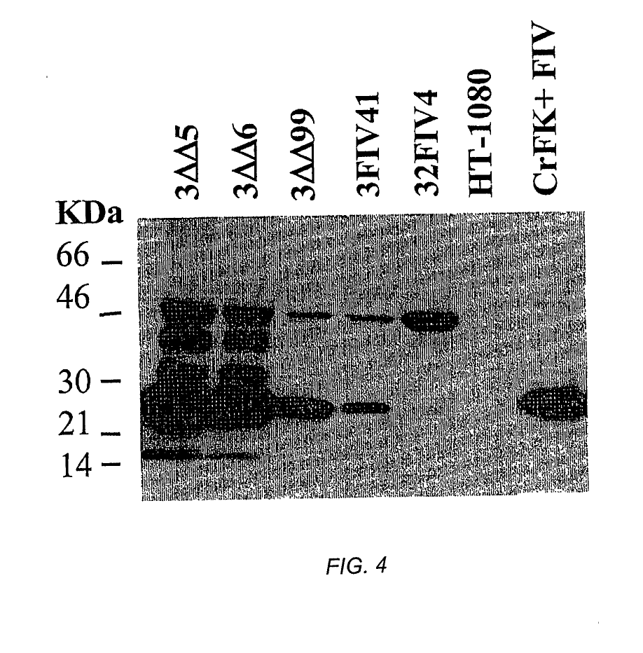

FIG. 5 is a graph depicting survival in two lots of Quidel pooled human serum of FIV-A and two different aliquots of FIV-G vector.

### DETAILED DESCRIPTION OF THE INVENTION

Prior to setting forth the invention, it may be helpful to an understanding thereof to first set forth definitions of certain terms that will be used hereinafter.

“Chimeric retroviral vector construct”, “MLV/FIV chimeric construct,” and “recombinant MLV/FIV vector” refers to a nucleic acid construct which carries sequences derived from MLV and from FIV. Preferably, FIV vector constructs, as described herein, are inserted into an MLV vector backbone. The MLV vector backbone includes 5′ and 3′ MLV LTRs, an MLV packaging signal and an MLV origin of second strand DNA synthesis. Within certain embodiments of the invention, the MLV portion of the chimeric construct is capable of producing, at high titers, vector particles which include MLV and FIV sequences. As described below, the FIV component of the chimeric construct is capable of directing the expression of a sequence(s) or gene(s) of interest.

A “nucleic acid” molecule can include, but is not limited to, procaryotic sequences, eucaryotic mRNA, cDNA from eucaryotic mRNA, genomic DNA sequences from eucaryotic (e.g., mammalian) DNA, and even synthetic DNA sequences. The term also captures sequences that include any of the known base analogs of DNA and RNA. For the purpose of describing the relative position of nucleotide sequences in a particular nucleic acid molecule throughout the instant application, such as when a particular nucleotide sequence is described as being situated “upstream,” “downstream,” “3′,” or “5“′ relative to another sequence, it is to be understood that it is the position of the sequences in the “sense” or “coding” strand of a DNA molecule that is being referred to as is conventional in the art.

A “gene” refers to a polynucleotide containing at least one open reading frame that is capable of encoding a particular polypeptide or protein after being transcribed or translated. Any of the polynucleotide sequences described herein may be used to identify larger fragments or full-length coding sequences of the genes with which they are associated. Methods of isolating larger fragment sequences are know to those of skill in the art.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting procaryotic microorganisms or eucaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

Two or more polynucleotide sequences can be compared by determining their “percent identity.” Two or more amino acid sequences likewise can be compared by determining their “percent identity.” The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An implementation of this algorithm for nucleic acid and peptide sequences is provided by the Genetics Computer Group (Madison, Wis.) in their BestFit utility application. The default parameters for this method are described in the Wisconsin Sequence Analysis Package Program Manual, Version 8 (1995) (available from Genetics Computer Group, Madison, Wis.). Other equally suitable programs for calculating the percent identity or similarity between sequences are generally known in the art.

For example, percent identity of a particular nucleotide sequence to a reference sequence can be determined using the homology algorithm of Smith and Waterman with a default scoring table and a gap penalty of six nucleotide positions. Another method of establishing percent identity in the context of the present invention is to use the MPSRCH package of programs copyrighted by the University of Edinburgh, developed by John F. Collins and Shane S. Sturrok, and distributed by IntelliGnetics, Inc. (Mountain View, Calif.). From this suite of packages, the Smith-Waterman algorithm can be employed where default parameters are used for the scoring table (for example, gap open penalty of 12, gap extension penalty of one, and a gap of six). From the data generated, the “Match” value reflects “sequence identity.” Other suitable programs for calculating the percent identity or similarity between sequences are generally known in the art, such as the alignment program BLAST, which can also be used with default parameters. For example, BLASTN and BLASTP can be used with the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

One of skill in the art can readily determine the proper search parameters to use for a given sequence in the above programs. For example, the search parameters may vary based on the size of the sequence in question. Thus, for example, a representative embodiment of the present invention would include an isolated polynucleotide having X contiguous nucleotides, wherein (i) the X contiguous nucleotides have at least about 50% identity to Y contiguous nucleotides derived from any of the sequences described herein, (ii) X equals Y, and (iii) X is greater than or equal to 6 nucleotides and up to 5000 nucleotides, preferably greater than or equal to 8 nucleotides and up to 5000 nucleotides, more preferably 10-12 nucleotides and up to 5000 nucleotides, and even more preferably 15-20 nucleotides, up to the number of nucleotides present in the full-length sequences described herein (e.g., see the Sequence Listing and claims), including all integer values falling within the above-described ranges.

A first polynucleotide is “derived from” second polynucleotide if it has the same or substantially the same basepair sequence as a region of the second polynucleotide, its cDNA, complements thereof, or if it displays sequence identity as described above. Similarly, a first polypeptide is “derived from” a second polypeptide if it is (i) encoded by a first polynucleotide derived from a second polynucleotide, or (ii) displays sequence identity to the second polypeptides as described above.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences which are immunologically identifiable with a polypeptide encoded by the sequence.

“Purified” or “isolated” when referring to a polynculeotide” refers to a polynucleotide of interest or fragment thereof which is essentially free, e.g., contains less than about 50%, preferably less than about 70%, and more preferably less than about 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

“FIV retroviral vector construct”, “FIV vector,” and “recombinant FIV vector” refers to a nucleic acid construct which carries, and within certain embodiments of the invention, is capable of directing the expression of a sequence(s) or gene(s) of interest. Briefly, the FIV vector must include at least one transcriptional promoter/enhancer or locus defining element(s), or other elements which control gene expression by other means such as alternative splicing, nuclear RNA export, post-translational modification of messenger, or post-transcriptional modification of protein. Such vector constructs must also include a packaging signal (preferably an FIV packaging signal), long terminal repeats (LTRs) or portion thereof, and positive and negative strand primer binding sites. Optionally, the recombinant FIV vector may also include a signal which directs polyadenylation, selectable and/or non-selectable markers, as well as one or more restriction sites and a translation termination sequence. Examples for selectable markers include but are not limited to neomycin (Neo), thymidin kinase (TK), hygromycin, phleomycin, puromycin, histidinol, green fluorescent protein (GFP), human placental alkaline phosphatase (PLAP) or DHFR. Examples for non-selectable markers are e.g. -galactosidase and human growth hormone (hGH). By way of example, such vectors typically include a 5′ FIV LTR, a tRNA binding site, a packaging signal, an origin of second strand DNA synthesis, and a 3′ FIV LTR.

A wide variety of heterologous sequences may be included within the vector construct, including for example, sequences which encode a protein (e.g., cytotoxic protein, disease-associated antigen, immune accessory molecule, or replacement gene), or which are useful as a molecule itself (e.g., as a ribozyme or antisense sequence). Alternatively, the heterologous sequence may merely be a “stuffer” or “filler” sequence, which is of a size sufficient to allow production of viral particles containing the RNA genome.

“Expression cassette” refers to an assembly which is capable of directing the expression of the sequence(s) or gene(s) of interest. The expression cassette must include a promoter or promoter/enhancer which, when transcribed, is operably linked to the sequence(s) or gene(s) of interest, as well as a polyadenylation sequence. Within certain embodiments of the invention, the expression cassette described herein may be contained within a plasmid construct. In addition to the components of the expression cassette, the plasmid construct may also include a bacterial origin of replication, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “virion,” or “recombinant virion” is defined herein as an infectious, replication-defective virus composed of an protein shell, encapsidating a heterologous nucleotide sequence of interest. Virions are produced in a suitable host cell which has had a chimeric or FIV vector and necessary accessory functions introduced therein. In this manner, the host cell is rendered capable of encoding polypeptides that are required for packaging the FIV vector (containing a recombinant nucleotide sequence of interest) into infectious recombinant virion particles for subsequent gene delivery.

The term “host cell” denotes, for example, microorganisms, yeast cells, insect cells, and mammalian cells, that can be, or have been, used as recipients of an AAV helper construct, an AAV vector plasmid, an accessory function vector, or other transfer DNA. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

A “selectable marker” or “reporter marker” refers to a nucleotide sequence included in a gene transfer vector that has no therapeutic activity, but rather is included to allow for simpler preparation, manufacturing, characterization or testing of the gene transfer vector.

“Packaging cell” refers to a cell which contains those elements necessary for production of infectious recombinant retrovirus which are lacking in a recombinant retroviral vector. Typically, such packaging cells contain one or more expression cassettes which are capable of expressing proteins which encode gag, pol and env-derived proteins. Packaging cells can also contain expression cassettes encoding one or more of vif rev, or ORF 2 in addition to gag/pol and env expression cassettes.

“Producer cell” or “Vector Producing Cell Line” (VCL) refers to a cell which contains all elements necessary for production of recombinant FIV vector particles. As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

“FIV vector particle” as utilized within the present invention refers to a viral particle which carries at least one gene of interest, and may also contain a selectable marker. The recombinant FIV particle is capable of reverse transcribing its genetic material into DNA and incorporating this genetic material into a host cell's DNA upon infection. FIV vector particles may have a lentiviral envelope, a non-lentiviral envelope (e.g., an ampho or VSV-G envelope), a chimeric envelope or a modified envelope (e.g., truncated envelopes or envelopes containing heterologous sequences).

The Long Terminal Repeats (“LTRs”) of most retrovriuses are subdivided into three elements, designated U5, R and U3. These elements contain a variety of signals which are responsible for the biological activity of a retrovirus, including for example, promoter and enhancer elements which are located within U3. The R region appears to play an important role during reverse transcription and furthermore contains the polyadenylation signal, and the U5 region containing sequences of importance in reverse transcription aid packaging of the retroviral genome. Additionally, the LTRs contain cis elements, the inverted repeats, important during the process of integration. LTRs may be readily identified in the provirus (integrated DNA form) due to their precise duplication at either end of the genome. As utilized herein, a 5′ FIV LTR should be understood to include a 5′ promoter/enhancer element to allow reverse transcription and integration of the DNA form of the vector. The 3′ FIV LTR should be understood to include a polyadenylation signal to allow reverse transcription and integration of the DNA form of the vector.

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

By “pharmaceutically acceptable” or “pharmacologically acceptable” is meant a material which is not biologically or otherwise undesirable, i.e., the material may be administered to an individual in a formulation or composition without causing any undesirable biological effects or interacting in a deleterious manner with any of the components of the composition in which it is contained.

As used herein, “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection). An “effective amount” is an amount sufficient to effect beneficial or desired results. An effective amount can be administered in one or more administrations, applications or dosages.

General Overview

The present invention provides novel chimeric lentiviral vector constructs which contain sequences derived from both MLV and FIV. In particular, the chimeric vector constructs contain an FIV vector, as defined above, in an MLV vector backbone which includes MLV LTRs. Further, the FIV LTRs are preferably hybrid in that up to 75% of the wildtype FIV LTR sequence is deleted and replaced by one or more viral or non-viral promoter/enhancer elements (e.g., other retroviral LTRs and/or non-retroviral promoters/enhancers such as the CMV promoter/enhancer or the SV40 promoter) similar to the hybrid LTRs described by Chang, et al., J Virology 67, 743-752, 1993; Finer, et al., Blood 83, 43-50, 1994 and Robinson, et al., Gene Therapy 2, 269-278, 1995. The chimeric MLV/FIV vector allow the generation of FIV PCLs. In one aspect, the FIV vector-containing chimeric vector is transiently transfected into a suitable host cell. The necessary packaging elements are provided (in trans) to these cells and chimeric MLV-FIV vector particles (virions) are produced, for example in the supernatant. The MLV-FIV virions can be concentrated and used to transduce FIV PCLs at high multiplicity of infection (moi). Under appropriate conditions, the FIV vector RNA is transcribed, the packaging signal specifically recognized and the FIV RNA (without MLV sequences) packaged into FIV vector particles with high efficiency.

Thus, as described herein, in certain embodiments, an FIV vector with the hybrid 5′ LTR (e.g. pVETLC) into an MLV vector derived from the pBA-5b construct. Vectors with three general structures can be created: (1) 5′ LTR5′ LTRCMVGENE OF INTEREST3′ LTRPPT3′ LTR; (2) 5′ LTR3′ LTRGENE OF INTERESTCMV5′ LTRPPT3′ LTR; and (3) 3′ LTRpart of gagCMVGENE OF INTERESTPPTCMV-promoter:R:U5.

Advantages of the present invention include, but are not limited to: (i) production of FIV vectors at high titers; (ii) production of stable FIV packaging cell lines; and (iii) providing efficient retroviral vectors. All publications cited are hereby incorporated by reference in their entireties herein.

FIV Vectors

FIV vectors suitable for use in the present invention may be readily constructed from a wide variety of FIV strains. Representative examples of FIV strains and molecular clones of such isolates include the Petaluma strain and its molecular clones FIV34TF10 and FIV14 (Olmsted et al., PNAS 86:8088-8092, 1989; Olmsted et al., PNAS 86:2448-2452, 1989; Talbot et al., PNAS 86:5743-5747, 1989), the San Diego strain and its molecular clone PPR (Phillips et al., J. Virology 64:4605-4613, 1990), the Japanese strains and their molecular clones FTM191CG and FIV-TM2 (Miyazawa et al., J. Virology 65:1572-1577, 1991) and the Amsterdam strain and its molecular clone 19K1 (Siebelink et al., J. Virology 66:1091-1097, 1992). Such FIV strains may either be obtained from feline isolates, or more preferably, from depositories or collections such as the American Type Culture Collection (ATTC, Rockville, Md.), or isolated from known sources using commonly available techniques. Representative examples of such FIV vector constructs are set forth in more detail below.

Any of the above FIV strains may be readily utilized in order to assemble or construct FIV gene delivery vehicles given the disclosure provided herein, and standard recombinant techniques (e.g., Sambrock et al, Molecular Cloning: A laboratory Manual, 2nd ed., Cold Spring Harbor Laboratory Press, 1989; Kunkle, PNAS 82:488, 1985). In addition, within certain embodiments of the invention, portions of the FIV gene delivery vehicles may be derived from different viruses. For example, within one embodiment of the invention, recombinant FIV vector LTRs may be partially derived or obtained from HIV, a packaging signal from SIV, and an origin of second strand synthesis from HIV-2. The FIV vector constructs nested within the MLV backbone typically contain 5′ and 3′ FIV LTRs, a tRNA binding site, an FIV packaging signal, and an origin of second strand DNA synthesis. Certain preferred recombinant FIV vector constructs which are provided herein also comprise one or more genes of interest, each of which is discussed in more detail below.

The tRNA binding site and origin of second strand DNA synthesis are important for a retrovirus to be biologically active, and may be readily identified by one of skill in the art. For example, tRNA binds to a retroviral tRNA binding site by Watson-Crick base pairing, and is carried with the retrovirus genome into a viral particle. The tRNA is then utilized as a primer for DNA synthesis by reverse transcriptase. The tRNA binding site may be readily identified based upon its location just downstream from the 5′ LTR. The FIV portion of the chimeric vectors described herein preferably make use of a tRNA binding site derived from FIV.

Similarly, the origin of second strand DNA synthesis is, as its name implies, important for the second strand DNA synthesis of a retrovirus. This region, which is also referred to as the poly-purine tract (PPT), is located just upstream of the 3′ LTR.

The retroviral packaging signal sequence directs packaging of viral genetic material into the viral particle. A major part of the packaging signal in FIV lies between the 5′ FIV LTR and the gag/pol sequence with the packaging signal likely overlapping in part with the 5′ area of the gag/pol sequence.

In addition, the FIV vectors have a nuclear transport element which, within one aspect of the invention is the FIV RRE (Rev-responsive element). Within another aspect of the invention, the nuclear transport element is not FIV RRE but a heterologous transport element. Representative examples of suitable heterologous nuclear transport elements include the Mason-Pfizer monkey virus constitutive transport element, the MPMV CTE (Bray et al., PNAS USA 91, 1256-1260, 1994), the Hepatitis B Virus posttranscriptional regulatory element, the HBV PRE (Huang et al., Mol. Cell. Biol. 13:7476-7486, 1993 and Huang et al., J. Virology 68:3193-3199, 1994), other lentiviral Rev-responsive elements (Daly et al., Nature 342:816-819, 1989 and Zapp et al., Nature 342:714-716, 1989) or the PRE element from the woodchuck hepatitis virus. Further nuclear transport elements include the element in Rous sarcoma virus (Ogert et al., J. Virology 70:3834-3843, 1996; Liu & Mertz, Genes & Dev. 9:1766-1789, 1995) and the element in the genome of simian retrovirus type 1 (Zolotukhin et al., J. Virology 68:7944-7952, 1994). Other potential elements include the elements in the histone gene (Kedes, Annu. Rev. Biochem. 48:837-870, 1970), the a interferon gene (Nagata et al., Nature 287:401-408, 1980), the adrenergic receptor gene (Koilka et al., Nature 329:75-79, 1987), and the c-Jun gene (Hattorie et al., Proc. Natl. Acad. Sci. USA 85:9148-9152, 1988).

Within one aspect of the invention, recombinant FIV vector constructs are provided which contain one or more multiple cloning sites and/or code for one or more marker genes such as the ones described above.

Within one aspect of the invention, recombinant FIV vector constructs are provided which lack both gag/pol and env coding sequences. As utilized herein, the phrase “lacks gag/pol or env coding sequences” should be understood to mean that the FIV vector contains less than 20, preferably less than 15, more preferably less than 10, and most preferably less than 8 consecutive nucleotides which are found in gag/pol or env genes, and in particular, within gag/pol or env expression cassettes that are used to construct packaging cell lines for the FIV vector construct. The production of FIV vector constructs lacking gag/pol or env sequences may be accomplished by partially eliminating the packaging signal and/or the use of a modified or heterologous packaging signal. Within other embodiments of the invention, FIV vector constructs are provided wherein the packaging signal that may extend into, or overlap with, FIV gag/pol sequence is modified (e.g., deleted, truncated or bases exchanged). Within other aspects of the invention, FIV vector constructs are provided which include the packaging signal that may extend beyond the start of the gag/pol gene. Within certain embodiments, the packaging signal that may extend beyond the start of the gag/pol gene is modified in order to contain one, two or more stop codons within the gag/pol reading frame. Most preferably, one of the stop codons eliminates the start site.

With another aspect of the invention, the FIV vector constructs are designed such that the internal promoter present in the gag sequence is disrupted. Using a chimeric MLV/FIV vector where in the FIV vector is nested in a reverse orientation could potentially decrease packaging efficiency because antisense RNAs to the MLV genome generated by both the hybrid FIV promoter in the 5′ LTR and the internal promoter. Therefore, in certain embodiments, the internal promoter can be inactivated, for example, by inserting an intron into its TATA box. The intron would be in the sense orientation with respect to the MLV direction of transcription. Additionally, the intron can contain a polyA signal to ensure the minimal length of the RNA molecules generated by the FIV hybrid promoter.

In certain embodiments, the FIV vector is constructed in which at least one of the wild-type U3 regions of the FIV LTR is replaced with a promoter/enhancer elements having high transcriptional activity in non-feline (e.g., human) cells. In certain embodiments, both of the flanking wild-type U3 regions will be replaced with a high transcriptional activity promoter. Suitable promoters are known to those of skill in the art and described herein. Further, it is to be understood that when both FIV LTR U3 regions include a heterologous promoter element, each region can contain the same heterologous promoter or, alternatively, a different heterologous promoter can be used in the two FIV LTR regions found in the construct. This construct allows for the generation of FIV packaging cell lines via high multiplicity transduction of VSV-G pseudotyped FIV vectors and to higher titer FIV VPLCs as compared to traditional techniques in which the provector is introduced by transfection.

The MLV Vector Backbone

Because the wild type FIV LTR is rather weak in non-feline cells, use of FIV vectors to generate gene delivery vehicles typically results in low quantities of genomic vector RNA and, accordingly, low titers of FIV vector packaging cell lines. The present invention overcomes this problem by producing high titer FIV vectors using chimeric MLV-FIV vectors in a two-step procedure. Briefly, FIV vectors are inserted into an MLV vector backbone to produce the chimeric MLV/FIV vectors described herein. The chimeric vectors are then transfected into cells containing the necessary elements in trans to package chimeric MLV-FIV vectors. The chimeric vectors can then be isolated (e.g., from the supernatant, concentrated and used to transduce FIV packaging cell lines (PCLs) at a high multiplicity of infection. The FIV PCLs will transcribe and package only FIV RNA, leaving the MLV backbone behind.

Certain elements are preferably found in the MLV vector backbone, including but not limited to, MLV LTRs (e.g., wild-type, hybrid or a combination thereof), a tRNA binding site (preferably derived from MLV), an origin of second strand DNA synthesis and a packaging signal (preferably derived from MLV). As described above for FIV vectors, the tRNA binding site and origin of second strand DNA synthesis are important for a retrovirus to be biologically active, and may be readily identified by one of skill in the art. Similarly, the MLV backbone preferably includes an MLV packaging signal, which facilitates high level expression of MLV vector particles which include the FIV vector. Thus, as depicted in the Figures and exemplifed herein, the MLV vector backbone typically includes, but is not necessarily limited to, an MLV 5′ LTR, an MLV packaging signal, and MLV poly purine tract (PPT) and an MLV 3′ LTR. In one embodiment, the MLV vector backbone is derived from pBA-5b (see, e.g., U.S. Ser. No. 08/643,411).

The chimeric vectors may be readily constructed by inserting an FIV vector (e.g., pVET_{L}C) into an MLV vector backbone (e.g., derived from pBA-5b) by standard cloning methods well-known in the art, for example as described in Sambrook et al. and Ausubel et al, supra. The FIV vector can either be inserted in the 5′->3′ orientation or in the inverse 3′→5′ orientation. Without being bound by one theory, it appears that when the FIV vector is inserted into the MLV backbone in the 5′ to 3′ orientation, further modifications of the FIV polyA signal in the LTR can be used in order to make it less efficient and, accordingly, increase production of full-length MLV vector genomic RNA can be expected. For example, the FIV PPT can be modified in order to make it less efficient and increase expected yield of full-length MLV vector genomic RNA. Such modifications are within the purview of a skilled artisan in view of the teachings herein.

The chimeric MLV-FIV vectors can then be used to generate FIV packaging cell lines, for example by transduction of VSV-G or amphotropic pseudotyped vector (described below). The production of MLV vectors (carrying the FIV vectors) is preferably carried out by transient transfection of a suitable host cell line, e.g., 293T cells. In order to produce high titers, the chimeric vector is co-transfected with the necessary trans acting elements, for example the retroviral structural gene products gag, pol and/or env. The resulting MLV-based vectors can be concentrated and FIV packaging cell lines (PCLs) transduced a high mulitplicity of infection. In the FIV PCL setting, the MLV-FIV vector RNA is transcribed and the FIV packaging signal specifically recognized. Thus, the FIV RNA is packaged into FIV vector particles while the MLV vector backbone is not packaged.

Promoters

Within certain embodiments of the invention, the FIV vector component includes viral promoters, preferably CMV or SV40 promoters and/or enhancers are utilized to drive expression of one or more genes of interest.

Within other aspects of the invention, the FIV vector portion is provided wherein tissue-specific promoters are utilized to drive expression of one or more genes of interest. For example, FIV vector particles of the invention can contain a liver specific promoter to maximize the potential for liver specific expression of the exogenous DNA sequence contained in the vectors. Preferred liver specific promoters include the hepatitis B X-gene promoter and the hepatitis B core protein promoter. These liver specific promoters are preferably employed with their respective enhancers. See also PCT Patent Publications WO 90/07936 and WO 91/02805 for a description of the use of liver specific promoters in FIV vector particles.

Within certain embodiments of the invention, the FIV vector constructs provided herein may be generated such that more than one gene of interest is expressed. This may be accomplished through the use of di- or oligo-cistronic cassettes (e.g., where the coding regions are separated by 120 nucleotides or less, see generally Levin et al., Gene 108:167-174, 1991), or through the use of Internal Ribosome Entry Sites (“IRES”).

Packaging/Producer Cell Lines

Packaging cell lines suitable for use with the above described recombinant MLV/FIV chimeric vector constructs may be readily prepared given the disclosure provided herein. Briefly, the parent cell line from which the packaging cell line is derived can be selected from a wide variety of mammalian cell lines, including for example, human cells, monkey cells, feline cells, dog cells, mouse cells, and the like. The packaging cell line will be selected according to the product one wishes to obtain. For example, where vector particles including MLV and FIV sequences are desired, the packaging cell line chosen will recognize the packaging signal included in the MLV vector backbone. Alternatively, when particles including FIV sequences only are desired, the packaging cell line should recognize that packaging signal included in the FIV vector portion of the chimeric construct. As noted above, high titers of FIV vector can be obtained using a two-step process. In particular, the chimeric construct is first packaged using MLV-appropriate PCL. These MLV-FIV particles can then be concentrated and, when packaged with a FIV-appropriate PCL, particles containing FIV sequences are obtained at high titers.

Within one embodiment of the invention, potential packaging cell line candidates are screened by isolating the human placental alkaline phosphatase (PLAP) gene from the N2-derived retroviral vector pBAAP, and inserting the gene into the FIV vector construct. To generate infectious virus, the construct is co-transfected, for example with a VSV-G encoding expression cassette (e.g., pMLP-G as described by Emi et al., J. Virology 65, 1202-1207, 1991; or pCMV-G, see U.S. Pat. No. 5,670,354) into 293 cells, and the virus harvested 48 hours after transfection. The resulting virus can be utilized to infect candidate host cells which are subsequently FACS-analyzed using antibodies specific for PLAP. Candidate host cells include, e.g. human cells such as HeLa (ATCC CCL 2.1), HT-1080 (ATCC CCL 121), 293 (ATCC CRL 1573), Jurkat (ATCC TIB 153), supT1 (NIH AIDS Research and Reference reagent program catalog #100), and CEM (ATCC CCL 119) or feline cells such as CrFK (ATCC CCL 94), G355-5 (Ellen et al., Virology 187:165-177, 1992), MYA-1 (Dahl et al., J. Virology 61:1602-1608, 1987) or 3201-B (Ellen et al., Virology 187:165-177, 1992). Production of p24 and reverse transcriptase can also be analyzed in the assessment of suitable packaging cell lines.

After selection of a suitable host cell for the generation of a packaging cell line, one or more expression cassettes are introduced into the cell line in order to complement or supply in trans components of the vector which have been deleted (see generally U.S. Ser. No. 08/240,030, filed May 9, 1994; see also U.S. Ser. No. 07/800,921, filed Nov. 27, 1991).

Representative examples of suitable expression cassettes include packaging expression cassettes and envelope expression cassettes which are described in more detail below. Briefly, packaging expression cassettes encode either gag/pol sequences alone, gag/pol sequences and one or more of vif, rev or ORF 2 or expression cassettes encoding one or more of vif, rev or ORF 2 alone. Envelope expression cassettes encode either an env sequence alone or env and one or more of vif, rev or ORF 2.

Utilizing the above-described expression cassettes, a wide variety of packaging cell lines can be generated. Any combination of the above mentioned expression cassettes can be used for the production of FIV-derived packaging cell lines. For example, within one aspect packaging cell lines are provided comprising an expression cassette that comprises a sequence encoding gag/pol, and a nuclear transport element, wherein the promoter is operably linked to the sequence encoding gag/pol.

Within other aspects, packaging cell lines are provided comprising a promoter and a sequence encoding ORF 2, vif, rev, or an envelope (e.g., amphotropic envelope or VSV-G), wherein the promoter is operably linked to the sequence encoding ORF 2, vif, rev, or, the envelope.

Within further embodiments, the packaging cell line may further comprise a sequence encoding any one or more of rev, ORF 2 or vif. For example, the packaging cell line may contain only ORF 2, vif, or rev alone, ORF 2 and vif, ORF 2 and rev, vif and rev or all three of ORF 2, vif and rev.

Within other aspects, the packaging cell line is derived from a feline or human parent cell, contains MLV- or FIV-derived packaging constructs always coding for a dUTPase and MLV, FIV, amphotropic or VSV-G derived env expression cassettes for the use to deliver nucleic acid sequences to cats.

Within another embodiment, the expression cassette is stably integrated. Within yet another embodiment, the packaging cell line, upon introduction of a chimeric vector, produces particles at a concentration of greater than 10³, 10⁴, 10⁵, 10⁶, 10⁷, 10⁸, or, 10⁹ cfu/ml. Within yet another embodiment, the packaging cell line, upon introduction of particles including the sequence of the chimeric vector, produces FIV-derived particles at a concentration of greater than 10³, 10⁴, 10⁵, 10⁶, 10⁷, 10⁸, or, 10⁹ cfu/ml. Within further embodiments the promoter is inducible. Within certain preferred embodiments of the invention, the packaging cell line, upon introduction of a chimeric vector or vector particles, produces particles that are free of replication competent virus.

Construction of Packaging Expression Cassettes

As noted above, the present invention provides a variety of packaging expression cassettes which, in combination with the env expression cassettes of the present invention, enable the construction of packaging cell lines. Further introduction of chimeric vector constructs into packaging cell lines enables the production of producer cell lines. The term “packaging expression cassettes” is used for expression constructs encoding gag/pol sequences alone, gag/pol and one or more of rev, vif or ORF 2 encoding sequences, or for constructs encoding for one or more of rev, vif or ORF 2 encoding sequences alone. Representative examples of suitable packaging expression cassettes include gag/pol expression cassettes which comprise a promoter and a sequence encoding gag/pol. Within another embodiment, the gag/pol expression cassette comprises a promoter, a sequence encoding gag/pol and at least one of rev, ORF 2 or vif wherein the promoter is operably linked to gag/pol and rev, vif or ORF 2.

Within further embodiments rev expression cassettes are provided comprising a promoter and a sequence encoding rev. Within another embodiment, the rev epxression cassette comprises a promoter, a sequence encoding rev and at least one of ORF 2 or vif, wherein the promoter is operably linked to rev and ORF 2 or vif

Briefly, FIV-derived gag/pol genes contain a gag region which encodes a variety of structural proteins that make up the core matrix, capsid and nucleocapsid, and a pol region which contains genes which encode (1) a protease for the processing of gag/pol and env proteins, (2) a reverse transcriptase polymerase, (3) an RNase H, (4) the enzyme deoxyuridine triphosphatase (dUTPase) and (5) an integrase, which is necessary for integration of the FIV provector into the host genome. Vif is a protein encoded by ORF l of FIV and believed to be the feline equivalent of the HIV viral infectivity factor, vif. Orf 2 of FIV corresponds roughly in size and location to Orf S of Visna Virus S which encodes a protein capable of some degree of transactivation (Davis et al., PNAS USA 86:414-418, 1989). Although FIV-derived gag/pol, rev, vif and/or ORF 2 genes may be utilized to construct the gag/pol expression cassettes of the present invention, a variety of other non-retroviral (and non-viral) genes may also be utilized to construct the gag/pol expression cassette. For example, a gene which encodes retroviral RNase H may be replaced with genes which encode bacterial (e.g., E. coli or Thermus thermophilus) RNase H. Similarly, the FIV integrase gene may be replaced by other genes with similar function (e.g., yeast retrotransposon TY3 integrase).

Within one embodiment of the invention, the gag/pol expression cassette contains a heterologous promoter, and/or heterologous polyadenylation sequence. As utilized herein, “heterologous” promoters or polyadenylation sequences refers to promoters or polyadenylation sequences which are from a different source from which the gag/pol gene (and preferably the env gene and FIV vector construct) is derived from. Representative examples of suitable promoters include the Cytomegalovirus Immediate Early (“CMV IE”) promoter, the Herpes Simplex Virus Thymidine Kinase (“HSVTK”) promoter, the Rous Sarcoma Virus (“RSV”) promoter, the Adenovirus major-late promoter and the SV 40 promoter. Representative examples of suitable polyadenylation signals include the SV 40 late polyadenylation signal, the SV40 early polyadenylation signal and the bovine growth hormone polyadenylation/termination signal.

Within one embodiment of the invention, a partial sequence of the gag/pol expression cassette containing the full sequence encoding for the enzyme dUTPase, is used as a packaging expression cassette.

Within another embodiment of the invention, one or more packaging expression constructs can be expressed from an inducible promoter system (e.g., the tet-inducible promoter system described by Bujard et al., PNAS 89, 5547-5551, 1992).

Within preferred aspects of the present invention, gag/pol expression cassettes such as those described above will not co-encapsidate along with a replication competent virus.

Construction of Envelope (env) Expression Cassettes

Within other aspects of the present invention, env expression cassettes are provided which, in combination with the packaging expression cassettes and vector constructs described above, enable the production of FIV vector particles and preclude formation of replication competent virus by homologous recombination. In addition, FIV viral particles described in this invention confer a particular specificity of the resultant vector particle (e.g., amphotropic, ecotropic, xenotropic, polytropic or pantropic). Briefly, in a wild-type FIV the env gene encodes two principal proteins, the surface glycoprotein “SU” and the transmembrane protein “TM”, which are translated as a polyprotein, and subsequently separated by proteolytic cleavage. Representative examples of the SU and TM proteins are the gp120 protein and gp41 protein in HIV, and the gp70 protein and p15e protein in MoMLV. In some retroviruses, a third protein designated the “R” peptide” of undetermined function, is also expressed from the env gene and separated from the polyprotein by proteolytic cleavage.

The term “env expression cassettes” is used for expression constructs encoding env sequences alone or env and one or more of rev, vif or ORF 2 encoding sequences.

A wide variety of env expression cassettes may be constructed given the disclosure provided herein, and utilized within the present invention to produce vector particles. Within one aspect of the present invention, env expression cassettes are provided comprising a promoter operably linked to an env gene, wherein preferably no more than 6, 8, 10, 15, or 20 consecutive retroviral nucleotides are included upstream (5′ ) of and/or contiguous with said env gene. Within other aspects of the invention, env expression cassettes are provided comprising a promoter operably linked to an env gene, wherein the env expression cassette does not contain a consecutive sequence of greater than 20, preferably less than 15, more preferably less than 10, and most preferably less than 8 or 6 consecutive nucleotides which are found in a gag/pol expression cassette, and in particular, in a gag/pol expression cassette that will be utilized along with the env expression cassette to create a packaging cell line.

Within another aspect of the present invention, env expression cassettes are provided which contain a heterologous promoter, a heterologous leader sequence and/or heterologous polyadenylation sequence. As utilized herein, “heterologous” promoters, leaders or polyadenylation sequences refers to sequences which are from a different source from which the env gene (and preferably the packaging expression constructs and FIV vector construct) is derived from. Representative examples of suitable promoters include the CMV IE promoter, the HSVTK promoter, the RSV promoter, the Adenovirus major-late promoter and the SV 40 promoters. Representative examples of suitable polyadenylation signals include the SV 40 late polyadenylation signal, the SV40 early polyadenylation signal, and the bovine growth hormone termination/polyadenylation sequence. Preferably any such termination/polyadenylation sequence will not have any 10 bp stretch which has more than 80% homology to a chimeric vector construct.

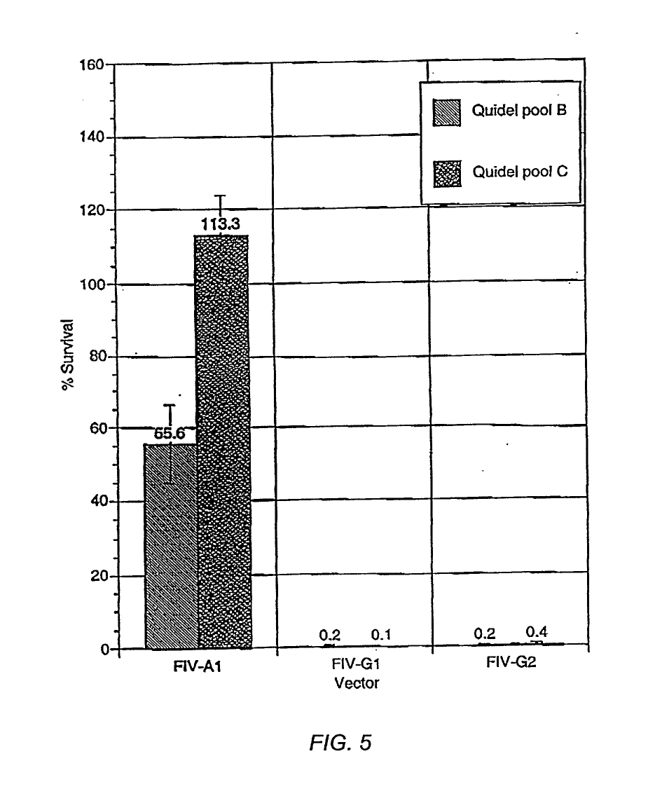

Chimeric MLV/FIV vectors can be pseudotyped with any suitable protein, for example VSV-G envelope or an amphotrophic envelope protein. As described in more detail below in Examples 6 and 7, FIV vectors alone can be pseudotyped at least with the VSV-G envelope protein. Based on this result it is evident that FIV may be pseudotyped with either the native or partially modified forms of heterologous envelope proteins. However, as detailed in Example 12, VSV-G pseudotyped MLV and FIV vectors produced in human cells appear to be inactivated by human serum complement. As shown in FIG. 5, MLV and FIV vectors containing amphotrophic envelope are resistant to human serum inactivation. Accordingly, as the use of complement resistant vectors will help make treatment more effective and efficient, in a preferred embodiment of the present invention, the chimeric MLV-FIV vectors (e.g., to produce either MLV-FIV virions or FIV virions alone) are pseudotyped with amphotrophic envelope. Therefore, in the present invention, the source of the viral env sequence may be derived from a wide range of retroviruses. For example, preferred envelope encoding sequences can be obtained from VSV (Vesicular Stomatitis Virus); amphotropic, ecotropic, polytropic or xenotropic MLV, HIV, FIV, or GaLV (Gibbon Ape Leukemia Virus), more preferably from amphotropic sources.

Within one embodiment of the invention, modified forms of env expression cassettes are provided. For example truncated HIV envelopes or hybrid envelopes are suitable for the production of FIV vector particles. Hybrid envelopes are understood to be env expression cassettes encoding viral envelopes plus heterologous viral or non-viral sequences that are added in addition or in place of viral env encoding sequences. Further, the env expression cassette may target the viral particle to a receptor of a particular cell type by linking the env coding sequences to an antibody or a particular ligand.

Within one embodiment of the invention, env expression cassettes are provided comprising a promoter and a sequence encoding a viral envelope sequence env alone. Within another embodiment of the invention, any of the above mentioned env expression cassettes are provided comprising a promoter, a sequence coding for env and at least one of rev, ORF 2 or vif, wherein the promoter is operably linked to env and rev, ORF 2 or vif.

Within another embodiment of this invention, any of the above described env expression cassettes can be expressed from an inducible promoter system (e.g., the tet-inducible promoter system described by Bujard et al., PNAS 89, 5547-5551, 1992).

Genes of Interest/Heterologous Nucleic Acid Molecules

A wide variety of nucleic acid molecules may be carried and/or expressed by the chimeric vectors and resulting FIV vector particles of the present invention. As used herein, “pathogenic agent” refers to a cell that is responsible for a disease state. Representative examples of pathogenic agents include tumor cells, autoreactive immune cells, hormone secreting cells, cells which lack a function that they would normally have, cells that have an additional inappropriate gene expression which does not normally occur in that cell type, and cells infected with bacteria, viruses, or other intracellular parasites. In addition, as used herein “pathogenic agent” may also refer to a cell that has become tumorigenic due to inappropriate insertion of nucleic acid molecules contained by the FIV vector into a host cell's genome.

Examples of nucleic acid molecules which may be carried and/or expressed by FIV vector particles of the present invention include genes and other nucleic acid molecules which encode a substance, as well as biologically active nucleic acid molecules such as inactivating sequences that incorporate into a specified intracellular nucleic acid molecule and inactivate that molecule. A nucleic acid molecule is considered to be biologically active when the molecule itself provides the desired benefit. For example, the biologically active nucleic acid molecule may be an inactivating sequence that incorporates into a specified intracellular nucleic acid molecule and inactivates that molecule, or the molecule may be a tRNA, rRNA or mRNA that has a configuration that provides a binding capability.

Substances which may be encoded by the nucleic acid molecules described herein include proteins (e.g., antibodies including single chain molecules), immunostimulatory molecules (such as antigens) immunosuppressive molecules, blocking agents, palliatives (such as toxins, antisense ribonucleic acids, ribozymes, enzymes, and other material capable of inhibiting a function of a pathogenic agent) cytokines, various polypeptides or peptide hormones, their agonists or antagonists, where these hormones can be derived from tissues such as the pituitary, hypothalamus, kidney, endothelial cells, liver, pancreas, bone, hemopoetic marrow, and adrenal. Such polypeptides can be used for induction of growth, regression of tissue, suppression of immune responses, apoptosis, gene expression, blocking receptor-ligand interaction, immune responses and can be treatment for certain anemias, diabetes, infections, high blood pressure, abnormal blood chemistry or chemistries (e.g., elevated blood cholesterol, deficiency of blood clotting factors, elevated LDL with lowered HDL), levels of Alzheimer associated amyloid protein, bone erosion/calcium deposition, and controlling levels of various metabolites such as steroid hormones, purines, and pyrimidines.

For palliatives, when “capable of inhibiting a function” is utilized within the context of the present invention, it should be understood that the palliative either directly inhibits the function or indirectly does so, for example, by converting an agent present in the cells from one which would not normally inhibit a function of the pathogenic agent to one which does. Examples of such functions for viral diseases include adsorption, replication, gene expression, assembly, and exit of the virus from infected cells. Examples of such functions for cancerous diseases include cell replication, susceptibility to external signals (e.g., contact inhibition), and lack of production of anti-oncogene proteins. Examples of such functions for cardiovascular disease include inappropriate growth or accumulation of material in blood vessels, high blood pressure, undesirable blood levels of factors such as cholesterol or low density lipoprotein that predispose to disease, localized hypoxia, and inappropriately high and tissue-damaging levels of free radicals. Examples of such functions for neurological conditions include pain, lack of dopamine production, inability to replace damaged cells, deficiencies in motor control of physical activity, inappropriately low levels of various peptide hormones derived from neurological tissue such as the pituitary or hypothalamus, accumulation of Alzheimer's Disease associated amyloid plaque protein, and inability to regenerate damaged nerve junctions. Examples of such functions for autoimmune or inflammatory disease include inappropriate production of cytokines and lymphokines, inappropriate production and existence of autoimmune antibodies and cellular immune responses, inappropriate disruption of tissues by proteases and collagenases, lack of production of factors normally supplied by destroyed cells, and excessive or aberrant regrowth of tissues under autoimmune attack.

Within one aspect of the present invention, methods are provided for administration of a recombinant FIV vector which directs the expression of a palliative. Representative examples of palliatives that act directly to inhibit the growth of cells include toxins such as ricin (Lamb et al., Eur. J. Biochem. 148:265-270, 1985), abrin (Wood et al., Eur. J. Biochem. 198:723-732,1991; Evensen et al., J. of Biol. Chem. 266:6848-6852, 1991; Collins et al., J. of Biol. Chem. 265:8665-8669, 1990; Chen et al., Fed. of Eur. Biochem Soc. 309:115-118, 1992), diphtheria toxin (Tweten et al., J. Biol. Chem. 260:10392-10394, 1985), cholera toxin (Mekalanos et al., Nature 306:551-557, 1983; Sanchez & Holmgren, PNAS 86:481-485, 1989), gelonin (Stirpe et al., J. Biol. Chem. 255:6947-6953, 1980), pokeweed (Irvin, Pharmac. Ther. 21:371-387, 1983), antiviral protein (Barbieri et al., Biochem. J 203:55-59, 1982; Irvin et al., Arch. Biochem. & Biophys. 200:418-425, 1980; Irvin, Arch. Biochem. & Biophys. 169:522-528, 1975), tritin, Shigella toxin (Calderwood et al., PNAS 84:4364-4368, 1987; Jackson et al., Microb. Path. 2:147-153, 1987), and Pseudomonas exotoxin A (Carroll and Collier, J. Biol. Chem. 262:8707-8711, 1987). A detailed description of recombinant retroviruses which express Russel's Viper Venom is provided in U.S. Ser. No. 08/368,574, filed Dec. 30, 1994.

Within other aspects of the invention, the FIV vector carries a gene specifying a product which is not in itself toxic, but when processed or modified by a protein, such as a protease specific to a viral or other pathogen, is converted into a toxic form. For example, recombinant retrovirus could carry a gene encoding a proprotein chain, which becomes toxic upon processing by the FIV protease. More specifically, a synthetic inactive proprotein form of the toxic ricin or diphtheria A chains could be cleaved to the active form by arranging for the FIV virally encoded protease to recognize and cleave off an appropriate “pro” element.

Within a related aspect of the present invention, FIV vectors are provided which direct the expression of a gene product(s) that activates a compound with little or no cytotoxicity into a toxic product. Briefly, a wide variety of gene products which either directly or indirectly activate a compound with little or no cytotoxicity into a toxic product may be utilized within the context of the present invention. Representative examples of such gene products include HSVTK and VZVTK which selectively monophosphorylate certain purine arabinosides and substituted pyrimidine compounds, converting them to cytotoxic or cytostatic metabolites. More specifically, exposure of the drugs ganciclovir, acyclovir, or any of their analogues (e.g., FIAC, DHPG) to HSVTK, phosphorylates the drug into its corresponding active nucleotide triphosphate form.

In a manner similar to the preceding embodiment, FIV vectors may be generated which carry a gene for phosphorylation, phosphoribosylation, ribosylation, or other metabolism of a purine- or pyrimidine-based drug. Such genes may have no equivalent in mammalian cells, and might come from organisms such as a virus, bacterium, fungus, or protozoan. Representative examples include: E. coli guanine phosphoribosyl transferase (“gpt”) gene product, which converts thioxanthine into thioxanthine monophosphate (see Besnard et al., Mol. Cell. Biol. 7:4139-4141, 1987); alkaline phosphatase, which will convert inactive phosphorylated compounds such as mitomycin phosphate and doxorubicin-phosphate to toxic dephosphorylated compounds; fungal (e.g., Fusarium oxysporum) or bacterial cytosine deaminase which will convert 5-fluorocytosine to the toxic compound 5-fluorouracil (Mullen, PNAS 89:33, 1992); carboxypeptidase G2 which will cleave the glutamic acid from para-N-bis(2-chloroethyl) aminobenzoyl glutamic acid, thereby creating a toxic benzoic acid mustard; and Penicillin-V amidase, which will convert phenoxyacetabide derivatives of doxorubicin and melphalan to toxic compounds.

Conditionally lethal gene products of this type have application to many presently known purine- or pyrimidine-based anticancer drugs, which often require intracellular ribosylation or phosphorylation in order to become effective cytotoxic agents. The conditionally lethal gene product could also metabolize a nontoxic drug, which is not a purine or pyrimidine analogue, to a cytotoxic form (see Searle et al., Brit. J. Cancer 53:377-384, 1986).

Additionally, in the instance where the target pathogen is a mammalian virus, FIV vectors may be constructed to take advantage of the fact that mammalian viruses in general tend to have “immediate early” genes, which are necessary for subsequent transcriptional activation of other viral promoter elements. Gene products of this nature are excellent candidates for intracellular signals (or “identifying agents”) of viral infection. Thus, conditionally lethal genes transcribed from transcriptional promoter elements that are responsive to such viral “immediate early” gene products could specifically kill cells infected with any particular virus. Additionally, since the human and interferon promoter elements are transcriptionally activated in response to infection by a wide variety of nonrelated viruses, the introduction of vectors expressing a conditionally lethal gene product like HSVTK, for example, from these viral-responsive elements (VREs) could result in the destruction of cells infected with a variety of different viruses.

In another embodiment of the invention, FIV vectors are provided that produce substances such as inhibitor palliatives, that inhibit viral assembly. In this context, the recombinant retrovirus codes for defective gag, pot, env or other viral particle proteins or peptides which inhibit in a dominant fashion the assembly of viral particles. Such inhibition occurs because the interaction of normal subunits of the viral particle is disturbed by interaction with the defective subunits.

One way of increasing the effectiveness of inhibitory palliatives is to express inhibitory genes, such as viral inhibitory genes, in conjunction with the expression of genes which increase the probability of infection of the resistant cell by the virus in question. The result is a nonproductive “dead-end” event which would compete for productive infection events. In the specific case of FIV, a recombinant retrovirus may be administered that inhibits FIV replication (by expressing anti-sense tat, etc., as described above) and also overexpress proteins required for infection, such as CD4. In this way, a relatively small number of vector-infected FIV-resistant cells act as a “sink” or “magnet” for multiple nonproductive fusion events with free virus or virally infected cells.

In another embodiment of the invention, FIV vectors are provided for the expression substances such as inhibiting peptides or proteins specific for viral protease. Viral protease cleaves the viral gag and gag/pol proteins into a number of smaller peptides. Failure of this cleavage in all cases leads to complete inhibition of production of infectious retroviral particles. The HIV protease is known to be an aspartyl protease, and these are known to be inhibited by peptides made from amino acids from protein or analogues. FIV vectors that inhibit HIV will express one or multiple fused copies of such peptide inhibitors.

Administration of the FIV vectors discussed above should be effective against many virally linked diseases, cancers, or other pathogenic agents.

In yet another aspect, FIV vectors are provided which have a therapeutic effect by encoding one or more ribozymes (RNA enzymes) (Haseloff and Gerlach, Nature 334:585, 1989) which will cleave, and hence inactivate, RNA molecules corresponding to a pathogenic function. Since ribozymes function by recognizing a specific sequence in the target RNA and this sequence is normally 12 to 17 bp, this allows specific recognition of a particular RNA sequence corresponding to a pathogenic state, such as HIV tat, and toxicity is specific to such pathogenic state. Representative examples of suitable ribozymes include hammerhead ribozymes (see Rossi et al., Pharmac. Ther 50:245-254, 1991) and hairpin ribozymes (Hampel et al., Nucl. Acids Res. 18:299-304, 1990; U.S. Pat. No. 5,254,678) and Tetrahymena based ribozymes (U.S. Pat. No. 4,987,071). Additional specificity may be achieved in some cases by making this a conditional toxic palliative, as discussed above.

In still another aspect, FIV vectors are provided comprising a biologically active nucleic acid molecule that is an antisense sequence (an antisense sequence may also be encoded by a nucleic acid sequence and then produced within a host cell via transcription). Briefly, antisense sequences are designed to bind to RNA transcripts, and thereby prevent cellular synthesis of a particular protein, or prevent use of that RNA sequence by the cell.

Representative examples of such sequences include antisense thymidine kinase, antisense dihydrofolate reductase (Maher and Dolnick, Arch. Biochem. & Biophys. 253:214-220, 1987; Bzik et al., PNAS 84:8360-8364, 1987), antisense HER2 (Coussens et al., Science 230:1132-1139, 1985), antisense ABL (Fainstein et al., Oncogene 4:1477-1481, 1989), antisense Myc (Stanton et al., Nature 310:423-425, 1984) and antisense ras, as well as antisense sequences which block any of the enzymes in the nucleotide biosynthetic pathway. In other embodiments, the antisense sequence is selected from the group consisting of sequences which encode influenza virus, HIV, HSV, HPV, CMV, and HBV. The antisense sequence may also be an antisense RNA complementary to RNA sequences necessary for pathogenicity. Alternatively, the biologically active nucleic acid molecule may be a sense RNA (or DNA) complementary to RNA sequences necessary for pathogenicity.

Within a further embodiment of the invention antisense RNA may be utilized as an anti-tumor agent in order to induce a potent Class I restricted response. Briefly, in addition to binding RNA and thereby preventing translation of a specific mRNA, high levels of specific antisense sequences are believed to induce the increased expression of interferons (including gamma-interferon), due to the formation of large quantities of double-stranded RNA. The increased expression of gamma interferon, in turn, boosts the expression of MHC Class I antigens. Preferred antisense sequences for use in this regard include actin RNA, myosin RNA, and histone RNA. Antisense RNA which forms a mismatch with actin RNA is particularly preferred.

In another embodiment, FIV vectors of the invention express a surface protein that is itself therapeutically beneficial. For example, in the particular case of HIV, expression of the human CD4 protein specifically in HIV-infected cells may be beneficial in two ways:

1. Binding of CD4 to HIV env intracellularly could inhibit the formation of viable viral particles much as soluble CD4 has been shown to do for free virus, but without the problem of systematic clearance and possible immunogenicity, since the protein will remain membrane bound and is structurally identical to endogenous CD4 (to which the patient should be immunologically tolerant). 2. Since the CD4/HIV env complex has been implicated as a cause of cell death, additional expression of CD4 (in the presence of excess HIV-env present in HIV-infected cells) leads to more rapid cell death and thus inhibits viral dissemination. This may be particularly applicable to monocytes and macrophages, which act as a reservoir for virus production as a result of their relative refractility to HIV-induced cytotoxicity (which, in turn, is apparently due to the relative lack of CD4 on their cell surfaces).

Still further aspects of the present invention relate to FIV vectors capable of immunostimulation. Briefly, the ability to recognize and defend against foreign pathogens is essential to the function of the immune system. In particular, the immune system must be capable of distinguishing “self” from “nonself” (i.e., foreign), so that the defensive mechanisms of the host are directed toward invading entities instead of against host tissues. Cytolytic T lymphocytes (CTLs) are typically induced, or stimulated, by the display of a cell surface recognition structure, such as a processed, pathogen-specific peptide, in conjunction with a MHC class I or class II cell surface protein.

Diseases suitable to treatment include viral infections such as influenza virus, respiratory syncytial virus, HPV, HBV, HCV, EBV, HIV, HSV, FeLV, FIV, Hantavirus, HTLV I, HTLV II and CMV, cancers such as melanomas, renal carcinoma, breast cancer, ovarian cancer and other cancers, and heart disease.

In one embodiment, the invention provides methods for stimulating a specific immune response and/or inhibiting viral spread by using FIV vectors that direct the expression of an antigen or modified form thereof in susceptible target cells, wherein the antigen is capable of either (1) initiating an immune response to the viral antigen or (2) preventing the viral spread by occupying cellular receptors required for viral interactions. Expression of the protein may be transient or stable with time. Where an immune response is to be stimulated to a pathogenic antigen, the FIV vector is preferably designed to express a modified form of the antigen which will stimulate an immune response and which has reduced pathogenicity relative to the native antigen. This immune response is achieved when cells present antigens in the correct manner, i.e., in the context of the MHC class I and/or II molecules along with accessory molecules such as CD3, ICAM-1, ICAM-2, LFA-1, or analogs thereof (e.g., Altmann et al., Nature 338:512, 1989). An immune response can also be achieved by transferring to an appropriate immune cell (such as a T lymphocyte) (a) the gene for the specific T-cell receptor that recognizes the antigen of interest (in the context of an appropriate MHC molecule if necessary), (b) the gene for an immunoglobulin which recognizes the antigen of interest, or (c) the gene for a hybrid of the two which provides a CTL response in the absence of the MHC context. Thus, recombinant retroviruses may also be used as an immunostimulant, immunomodulator, or vaccine, etc.

In the particular case of disease caused by HIV infection, where immunostimulation is desired, the antigen generated from a recombinant retrovirus may be in a form which will elicit either or both an HLA class I- or class II-restricted immune response. In the case of HIV envelope antigen, for example, the antigen is preferably selected from gp 160, gp 120, and gp 41, which have been modified to reduce their pathogenicity. In particular, the selected antigen is modified to reduce the possibility of syncytia, to avoid expression of epitopes leading to a disease enhancing immune response, to remove immunodominant, but haplotype-specific epitopes or to present several haplotype-specific epitopes, and allow a response capable of eliminating cells infected with most or all strains of HIV. The haplotype-specific epitopes can be further selected to promote the stimulation of an immune response within an animal which is cross-reactive against other strains of HIV. Antigens from other HIV genes or combinations of genes, such as gag, pol, rev, vif, nef, prot, gag/pol, gag prot, etc., may also provide protection in particular cases. HIV is only one example. This approach may be utilized for many virally linked diseases or cancers where a characteristic antigen (which does not need to be a membrane protein) is expressed. Representative examples of such “disease-associated” antigens all or portions of various eukaryotic (including for example, parasites), prokaryotic (e.g., bacterial) or viral pathogens. Representative examples of viral pathogens include the Hepatitis B Virus (“HBV”) and Hepatitis C Virus (“HCV”; see U.S. Ser. No. 08/102/132), Human Papiloma Virus (“HPV”; see WO 92/05248; WO 90/10459; EPO 133,123), Epstein-Barr Virus (“EBV”; see EPO 173,254; JP 1,128,788; and U.S. Pat. Nos. 4,939,088 and 5,173,414), Feline Leukemia Virus (“FeLV”; see U.S. Ser. No. 07/948,358; EPO 377,842; WO 90/08832; WO 93/09238), Feline Immunodeficiency Virus (“FIV”; U.S. Pat. No. 5,037,753; WO 92/15684; WO 90/13573; and JP 4,126,085), HTLV I and II, and Human Immunodeficiency Virus (“HIV”; see U.S. Ser. No. 07/965,084).

In accordance with the immunostimulation aspects of the invention, substances which are carried and/or expressed by the FIV vectors of the present invention may also include “immunomodulatory factors,” many of which are set forth above. Immunomodulatory factors refer to factors that, when manufactured by one or more of the cells involved in an immune response, or, which when added exogenously to the cells, causes the immune response to be different in quality or potency from that which would have occurred in the absence of the factor. The factor may also be expressed from a non-recombinant retrovirus derived gene, but the expression is driven or controlled by the recombinant retrovirus. The quality or potency of a response may be measured by a variety of assays known to one of skill in the art including, for example, in vitro assays which measure cellular proliferation (e.g., ³H thymidine uptake), and in vitro cytotoxic assays (e.g., which measure ⁵¹Cr release) (see, Warner et al., AIDS Res. and Human Retroviruses 7:645-655, 1991). Immunomodulatory factors may be active both in vivo and ex vivo.

Representative examples of such factors include cytokines, such as IL-1, IL-2 (Karupiah et al., J. Immunology 144:290-298, 1990; Weber et al., J. Exp. Med. 166:1716-1733, 1987; Gansbacher et al., J. Exp. Med. 172:1217-1224, 1990; U.S. Pat. No. 4,738,927), IL-3, IL-4 (Tepper et al., Cell 57:503-512, 1989; Golumbek et al., Science 254:713-716, 1991; U.S. Pat. No. 5,017,691), IL-5, IL-6 (Brakenhof et al., J. Immunol. 139:4116-4121, 1987; WO 90/06370), IL-7 (U.S. Pat. No. 4,965,195), IL-8, IL-9, IL-10, IL-11, IL-12, IL-13 (Cytokine Bulletin, Summer 1994), IL-14 and IL-15, particularly IL-2, IL-4, IL-6, IL-12, and IL-13, alpha interferon (Finter et al., Drugs 42(5):749-765, 1991; U.S. Pat. No. 4,892,743; U.S. Pat. No. 4,966,843; WO 85/02862; Nagata et al., Nature 284:316-320, 1980; Familletti et al., Methods in Enz. 78:387-394, 1981; Twu et al., Proc. Natl. Acad. Sci. USA 86:2046-2050, 1989; Faktor et al., Oncogene 5:867-872, 1990), beta interferon (Seif et al., J. Virol. 65:664-671, 1991), gamma interferons (Radford et al., The American Society of Hepatology 2008-2015, 1991; Watanabe et al., PNAS 86:9456-9460, 1989; Gansbacher et al., Cancer Research 50:7820-7825, 1990; Maio et al., Can. Immunol. Immunother. 30:34-42, 1989; U.S. Pat. Nos. 4,762,791; 4,727,138), G-CSF (U.S. Pat. Nos. 4,999,291 and 4,810,643), GM-CSF (WO 85/04188), tumor necrosis factors (TNFs) (Jayaraman et al., J. Immunology 144:942-951, 1990), CD3 (Krissanen et al., Immunogenetics 26:258-266, 1987), ICAM-1 (Altman et al., Nature 338:512-514, 1989; Simmons et al., Nature 331:624-627, 1988), ICAM-2, LFA-1, LFA-3 (Wallner et al., J. Exp. Med. 166(4):923-932, 1987), MHC class I molecules, MHC class II molecules, B7.1-0.3, b₂-microglobulin (Parnes et al., PNAS 78:2253-2257, 1981), chaperones such as calnexin, MHC linked transporter proteins or analogs thereof (Powis et al., Nature 354:528-531, 1991). Immunomodulatory factors may also be agonists, antagonists, or ligands for these molecules. For example soluble forms of receptors can often behave as antagonists for these types of factors, as can mutated forms of the factors themselves.

The choice of which immunomodulatory factor to include within a FIV vector may be based upon known therapeutic effects of the factor, or, experimentally determined. For example, a known therapeutic effector in chronic hepatitis B infections is alpha interferon. This has been found to be efficacious in compensating a patient's immunological deficit, and thereby assisting recovery from the disease. Alternatively, a suitable immunomodulatory factor may be experimentally determined. Briefly, blood samples are first taken from patients with a hepatic disease. Peripheral blood lymphocytes (PBLs) are restimulated in vitro with autologous or HLA matched cells (e.g., EBV transformed cells) that have been transduced with a recombinant retrovirus which directs the expression of an immunogenic portion of a hepatitis antigen and the immunomodulatory factor. These stimulated PBLs are then used as effectors in a CTL assay with the HLA matched transduced cells as targets. An increase in CTL response over that seen in the same assay performed using HLA matched stimulator and target cells transduced with a vector encoding the antigen alone, indicates a useful immunomodulatory factor. Within one embodiment of the invention, the immunomodulatory factor gamma interferon is particularly preferred.

The present invention also includes FIV vectors which encode immunogenic portions of desired antigens including, for example, viral, bacterial or parasite antigens. For example, at least one immunogenic portion of a hepatitis B antigen can be incorporated into an FIV vector. The immunogenic portion(s) which are incorporated into the FIV vector may be of varying length, although it is generally preferred that the portions be at least 9 amino acids long, and may include the entire antigen. Immunogenicity of a particular sequence is often difficult to predict, although T cell epitopes may be predicted utilizing the HLA A2. 1 motif described by Falk et al. (Nature 351:290, 1991). From this analysis, peptides may be synthesized and used as targets in an in vitro cytotoxic assay. Other assays, however, may also be utilized, including, for example, ELISA which detects the presence of antibodies against the newly introduced vector, as well as assays which test for T helper cells, such as gamma-interferon assays, IL-2 production assays, and proliferation assays.

Within one embodiment of the present invention, at least one immunogenic portion of a hepatitis C antigen can be incorporated into an FIV vector. Preferred immunogenic portion(s) of hepatitis C may be found in the C and NS3-NS4 regions since these regions are the most conserved among various types of hepatitis C virus (Houghton et al., Hepatology 14:381-388, 1991). Particularly preferred immunogenic portions may be determined by a variety of methods. For example, as noted above for the hepatitis B virus, identification of immunogenic portions of the polypeptide may be predicted based upon amino acid sequence. Briefly, various computer programs which are known to those of ordinary skill in the art may be utilized to predict CTL epitopes. For example, CTL epitopes for the HLA A2.1 haplotype may be predicted utilizing the HLA A2.1 motif described by Falk et al. (Nature 351:290, 1991). From this analysis, peptides are synthesized and used as targets in an in vitro cytotoxic assay.

Other disease-associated antigens which may be carried by the gene delivery constructs of the present invention include, for example immunogenic, non-tumorigenic forms of altered cellular components which are normally associated with tumor cells (see U.S. Ser. No. 08/104,424). Representative examples of altered cellular components which are normally associated with tumor cells include ras* (wherein * is understood to refer to antigens which have been altered to be non-tumorigenic), p53*, Rb*, altered protein encoded by Wilms' tumor gene, ubiquitin, mucin, protein encoded by the DCC. APC, and MCC genes, as well as receptors or receptor-like structures such as neu, thyroid hormone receptor, Platelet Derived Growth Factor (“PDGF”) receptor, insulin receptor, Epidermal Growth Factor (“EGF”) receptor, and the Colony Stimulating Factor (“CSF”) receptor.

Immunogenic portions of the disease-associated antigens described herein may be selected by a variety of methods. For example, the HLA A2.1/Kb transgenic mouse has been shown to be useful as a model for human T-cell recognition of viral antigens. Briefly, in the influenza and hepatitis B viral systems, the murine T-cell receptor repertoire recognizes the same antigenic determinants recognized by human T-cells. In both systems, the CTL response generated in the HLA A2.1/K^{b} transgenic mouse is directed toward virtually the same epitope as those recognized by human CTLs of the HLA A2.1 haplotype (Vitiello et al., J. Exp. Med. 173:1007-1015, 1991; Vitiello et al., Abstract of Molecular Biology of Hepatitis B Virus Symposia, 1992).

Immunogenic proteins of the present invention may also be manipulated by a variety of methods known in the art, in order to render them more immunogenic. Representative examples of such methods include: adding amino acid sequences that correspond to T helper epitopes; promoting cellular uptake by adding hydrophobic residues; by forming particulate structures; or any combination of these (see generally, Hart, op. cit., Milich et al., Proc. Natl. Acad. Sci. USA 85:1610-1614, 1988; Willis, Nature 340:323-324, 1989; Griffiths et al., J. Virol. 65:450-456, 1991).

Sequences which encode the above-described nucleic acid molecules may be obtained from a variety of sources. For example, plasmids which contain sequences that encode altered cellular products may be obtained from a depository such as the American Type Culture Collection (ATCC, Rockville, Md.), or from commercial sources such as Advanced Biotechnologies (Columbia, Md.). Representative examples of plasmids containing some of the above-described sequences include ATCC No. 41000 (containing a G to T mutation in the 12th codon of ras), and ATCC No. 41049 (containing a G to A mutation in the 12th codon).

Other nucleic acid molecules that encode the above-described substances, as well as other nucleic acid molecules that are advantageous for use within the present invention, may be readily obtained from a variety of sources, including for example depositories such as the American Type Culture Collection (ATCC, Rockville, Md.), or from commercial sources such as British Bio-Technology Limited (Cowley, Oxford England). Representative examples include BBG 12 (containing the GM-CSF gene coding for the mature protein of 127 amino acids), BBG 6 (which contains sequences encoding gamma interferon), ATCC No. 39656 (which contains sequences encoding TNF), ATCC No. 20663 (which contains sequences encoding alpha interferon), ATCC Nos. 31902, 31902 and 39517 (which contains sequences encoding beta interferon), ATCC No 67024 (which contains a sequence which encodes Interleukin-1b), ATCC Nos. 39405, 39452, 39516, 39626 and 39673 (which contains sequences encoding Interleukin-2), ATCC Nos. 59399, 59398, and 67326 (which contain sequences encoding Interleukin-3), ATCC No. 57592 (which contains sequences encoding Interleukin-4), ATCC Nos. 59394 and 59395 (which contain sequences encoding Interleukin-5), and ATCC No. 67153 (which contains sequences encoding Interleukin-6).

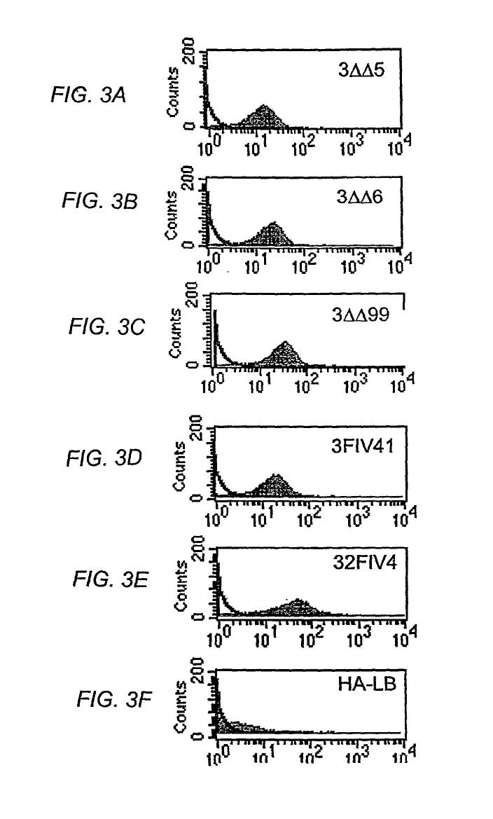

Molecularly cloned genomes which encode the hepatitis B virus may be obtained from a variety of sources including, for example, the American Type Culture Collection (ATCC, Rockville, Md.). For example, ATCC No. 45020 contains the total genomic DNA of hepatitis B (extracted from purified Dane particles) (see FIG. 3 of Blum et al., TIG 5(5):154-158, 1989) in the BamHI site of pBR322 (Moriarty et al., Proc. Natl. Acad. Sci. USA 78:2606-2610, 1981). (Note that correctable errors occur in the sequence of ATCC No. 45020.)

Alternatively, cDNA sequences for use with the present invention may be obtained from cells which express or contain the sequences. Briefly, within one embodiment mRNA from a cell which expresses the gene of interest is reverse transcribed with reverse transcriptase using oligo dT or random primers. The single stranded cDNA may then be amplified by PCR (see U.S. Pat. Nos. 4,683,202, 4,683,195 and 4,800,159). See also PCR Technology: Principles and Applications for DNA Amplification, Erlich (ed.), Stockton Press, 1989) utilizing oligonucleotide primers complementary to sequences on either side of desired sequences. In particular, a double stranded DNA is denatured by heating in the presence of heat stable Taq polymerase, sequence specific DNA primers, ATP, CTP, GTP and TTP. Double-stranded DNA is produced when synthesis is complete. This cycle may be repeated many times, resulting in a factorial amplification of the desired DNA.

Nucleic acid molecules which are carried and/or expressed by the FIV vectors described herein may also be synthesized, for example, on an Applied Biosystems Inc. DNA synthesizer (e.g., APB DNA synthesizer model 392 (Foster City, Calif.).

Methods for Utilizing the MLV/FIV Chimeric Vector Particles

As noted above, the present invention also provides methods for delivering a selected heterologous sequence to a vertebrate or insect, comprising the step of administering to a vertebrate or insect an FIV vector particle (e.g., an FIV vector particle produced from a chimeric vector as described herein) which is capable of expressing the selected heterologous sequence. Such FIV vector particles may be administered either directly (e.g., intravenously, intramuscularly, intraperitoneally, subcutaneously, orally, rectally, intraocularly, intranasally), or by various physical methods such as lipofection (Felgner et al., Proc. Natl. Acad. Sci. USA 84:7413-7417, 1989), direct DNA injection (Fung et al., Proc. Natl. Acad. Sci. USA 80:353-357, 1983; Seeger et al., Proc. Natl. Acad. Sci. USA 81:5849-5852; Acsadi et al., Nature 352:815-818, 1991); microprojectile bombardment (Williams et al., PNAS 88:2726-2730, 1991); liposomes of several types (see, e.g., Wang et al., PNAS 84:7851-7855, 1987); CaPO₄ (Dubensky et al., PNAS 81:7529-7533, 1984); DNA ligand (Wu et al., J. Biol Chem. 264:16985-16987, 1989); administration of nucleic acids alone (WO 90/11092); or administration of DNA linked to killed adenovirus (Curiel et al., Hum. Gene Ther. 3:147-154, 1992); via polycation compounds such as polylysine, utilizing receptor specific ligands; as well as with psoralen inactivated viruses such as Sendai or Adenovirus. In addition, the FIV vector particles may either be administered directly (i.e., in vivo), or to cells which have been removed (ex vivo), and subsequently returned.

As discussed in more detail below, FIV vector particles may be administered to a vertebrate or insect organism or cell for a wide variety of both therapeutic or productive purposes, including for example, for the purpose of stimulating a specific immune response; inhibiting the interaction of an agent with a host cell receptor; to express a toxic palliative, including for example, conditional toxic palliatives; to immunologically regulate the immune system; to express markers, for replacement gene therapy and/or to produce a recombinant protein. These and other uses are discussed in more detail below.

1. Immunostimulation

Within one aspect of the present invention, compositions and methods are provided for administering an FIV vector particle which is capable of preventing, inhibiting, stabilizing or reversing infectious, cancerous, auto-immune or immune diseases. Representative examples of such diseases include viral infections such as HIV, HBV, HCV, HTLV I, HTLV II, CMV, EBV, FIV and HPV, melanomas, diabetes, graft vs. host disease, Alzheimer's disease and heart disease. More specifically, within one aspect of the present invention, compositions and methods are provided for stimulating an immune response (either humoral or cell-mediated) to a pathogenic agent, such that the pathogenic agent is either killed or inhibited. Representative examples of pathogenic agents include bacteria, fungi, parasites, viruses and cancer cells.

Within one embodiment of the invention the pathogenic agent is a virus, and methods are provided for stimulating a specific immune response and inhibiting viral spread by using an FIV vector particle that directs the expression of an antigen or modified form thereof to susceptible target cells capable of either (1) initiating an immune response to the viral antigen or (2) preventing the viral spread by occupying cellular receptors required for viral interactions. Expression of the vector nucleic acid encoded protein may be transient or stable with time. Where an immune response is to be stimulated to a pathogenic antigen, the FIV vector is preferably designed to express a modified form of the antigen which will stimulate an immune response and which has reduced pathogenicity relative to the native antigen. This immune response is achieved when cells present antigens in the correct manner, i.e., in the context of the MHC class I and/or II molecules along with accessory molecules such as CD3, ICAM-1, ICAM-2, LFA-1, or analogues thereof (e.g., Altmann et al., Nature 338:512, 1989). Cells infected with FIV vector particles are expected to do this efficiently because they closely mimic genuine viral infection and because they: (a) are able to infect non-replicating cells, (b) integrate into the host cell genome, (c) are not associated with any life threatening human diseases. Because of these differences, FIV vectors can easily be thought of as safe viral vectors which can be used on healthy individuals for vaccine use.

This aspect of the invention has a further advantage over other systems that might be expected to function in a similar manner, in that the presenter cells are fully viable and healthy, and low levels of viral antigens, relative to heterologous genes, are expressed. This presents a distinct advantage since the antigenic epitopes expressed can be altered by selective cloning of sub-fragments of the gene for the antigen into an FIV vector particle, leading to responses against immunogenic epitopes which may otherwise be overshadowed by immunodominant epitopes. Such an approach may be extended to the expression of a peptide having multiple epitopes, one or more of the epitopes being derived from different proteins. Further, this aspect of the invention allows efficient stimulation of cytotoxic T lymphocytes (CTL) directed against antigenic epitopes, and peptide fragments of antigens encoded by sub-fragments of genes, through intracellular synthesis and association of these peptide fragments with MHC Class I molecules. This approach may be utilized to map major immunodominant epitopes for CTL induction.

An immune response may also be achieved by transferring to an appropriate immune cell (such as a T lymphocyte) the gene for the specific T cell receptor which recognizes the antigen of interest (in the context of an appropriate MHC molecule if necessary), for an immunoglobulin which recognizes the antigen of interest, or for a hybrid of the two which provides a CTL response in the absence of the MHC context. Thus, the FIV vector particles may be used as an immunostimulant, immunomodulator, or vaccine.

In one embodiment of the invention, the FIV vector particles are delivered to dendritic cells which are the most efficient antigen-presenting cells (APC) of the immune system. In contrast to other APCs, dendritic cells are known to elicit potent primary immune responses involving naive T-cells (Weissman et al., Clin. Microbiol. Rev. 10, 358-367, 1997). The transduction of dendritic cells with FIV vector particles encoding viral or cancer immunogens may initiate a strong immune response that might be efficacious in the fight of chronic viral diseases or certain types of cancers.

In another embodiment of the invention, methods are provided for producing inhibitor palliatives wherein FIV vector particles deliver and express defective interfering viral structural proteins, which inhibit viral assembly. Such FIV vector particles may encode defective gag, pol, env or other viral particle proteins or peptides and these would inhibit in a dominant fashion the assembly of viral particles. This occurs because the interaction of normal subunits of the viral particle is disturbed by interaction with the defective subunits.

In another embodiment of the invention, methods are provided for the expression of inhibiting peptides or proteins specific for viral protease. Briefly, viral protease cleaves the viral gag and gag/pol proteins into a number of smaller peptides. Failure of this cleavage in all cases leads to complete inhibition of production of infectious retroviral particles. As an example, the HIV protease is known to be an aspartyl protease and these are known to be inhibited by peptides made from amino acids from protein or analogues. FIV vectors to inhibit HIV will express one or multiple fused copies of such peptide inhibitors.

Another embodiment involves the delivery of suppressor genes which, when deleted, mutated, or not expressed in a cell type, lead to tumorigenesis in that cell type. Reintroduction of the deleted gene by means of an FIV vector particle leads to regression of the tumor phenotype in these cells. Examples of such cancers are retinoblastoma and Wilms Tumor. Since malignancy can be considered to be an inhibition of cellular terminal differentiation compared with cell growth, administration of the FIV vector particle and expression of gene products which lead to differentiation of a tumor should also, in general, lead to regression.

In yet another embodiment, the FIV vector provides a therapeutic effect by transcribing a ribozyme (an RNA enzyme) (Haseloff and Gerlach, Nature 334:585, 1989) which will cleave and hence inactivate RNA molecules corresponding to a pathogenic function. Since ribozymes function by recognizing a specific sequence in the target RNA and this sequence is normally 12 to 17 bp, this allows specific recognition of a particular RNA species such as a RNA or a retroviral genome. Additional specificity may be achieved in some cases by making this a conditional toxic palliative (see below). One way of increasing the effectiveness of inhibitory palliatives is to express viral inhibitory genes in conjunction with the expression of genes which increase the probability of infection of the resistant cell by the virus in question. The result is a nonproductive “dead-end” event which would compete for productive infection events. In the specific case of HIV, FIV vector particles may be delivered which inhibit HIV replication (by expressing anti-sense tat, etc., as described above) and also overexpress proteins required for infection, such as CD4. In this way, a relatively small number of vector-infected HIV-resistant cells act as a “sink” or “magnet” for multiple nonproductive fusion events with free virus or virally infected cells.

2. Blocking Agents

Many infectious diseases, cancers, autoimmune diseases, and other diseases involve the interaction of viral particles with cells, cells with cells, or cells with factors produced by themselves or other cells. In viral infections, viruses commonly enter cells via receptors on the surface of susceptible cells. In cancers or other proliferative conditions (e.g., restenosis), cells may respond inappropriately or not at all to signals from other cells or factors, or specific factors may be mutated, overexpressed, or underexpressed, resulting in loss of appropriate cell cycle control. In autoimmune disease, there is inappropriate recognition of “self” markers. Within the present invention, such interactions may be blocked by producing, in vivo, an analogue to either of the partners in an interaction. Alternatively, cell cycle control may be restored by preventing the transition from one phase to another (e.g., G1 to S phase) using a blocking factor which is absent or underexpressed. This blocking action may occur intracellularly, on the cell membrane, or extracellularly, and the action of the FIV vector particle carrying a gene for a blocking agent, can be mediated either from inside a susceptible cell or by secreting a version of the blocking protein to locally block the pathogenic interaction.

In the case of HIV, the two agents of interaction are the gp 120/gp 41 envelope protein and the CD4 receptor molecule. Thus, an appropriate blocker would be an FIV vector expressing either an HIV env analogue that blocks HIV entry without causing pathogenic effects, or a CD4 receptor analogue. The CD4 analogue would be secreted and would function to protect neighboring cells, while the gp 120/gp 41 is secreted or produced only intracellularly so as to protect only the vector-containing cell. It may be advantageous to add human immunoglobulin heavy chains or other components to CD4 in order to enhance stability or complement lysis. Administration of an FIV vector particle encoding such a hybrid-soluble CD4 to a host results in a continuous supply of a stable hybrid molecule. Efficacy of treatment can be assayed by measuring the usual indicators of disease progression, including antibody level, viral antigen production, infectious HIV levels, or levels of nonspecific infections.

In the case of uncontrolled proliferative states, such as cancer or restenosis, cell cycle progression may be halted by the expression of a number of different factors that affect signaling by cyclins or cyclin-dependent kinases (CDK). For example, the cyclin-dependent kinase inhibitors, p16, p21, and p27 each regulate cyclin:CDK mediated cell cycle signaling. Overexpression of these factors within a cell by a FIV vector particle results in a cytostatic suppression of cell proliferation. Other factors that may be used therapeutically, as blocking agents or targets, include, for example, wild-type or mutant Rb, p53, Myc, Fos, Jun, PCNA, GAX, and p15.

3. Expression of Palliatives

Techniques similar to those described above can be used to produce FIV vector particles which direct the expression of an agent (or “palliative”) which is capable of inhibiting a function of a pathogenic agent or gene. Within the present invention, “capable of inhibiting a function” means that the palliative either directly inhibits the function or indirectly does so, for example, by converting an agent present in the cells from one which would not normally inhibit a function of the pathogenic agent to one which does. Examples of such functions for viral diseases include adsorption, replication, gene expression, assembly, and exit of the virus from infected cells. Examples of such functions for a cancerous cell, cancer-promoting growth factor, or uncontrolled proliferative condition (e.g., restenosis) include viability, cell replication, altered susceptibility to external signals (e.g., contact inhibition), and lack of production or production of mutated forms of anti-oncogene proteins.

a. Inhibitor Palliatives

In one aspect of the present invention, the FIV vector particle directs the expression of a gene which can interfere with a function of a pathogenic agent, for instance in viral or malignant diseases. Such expression may either be essentially continuous or in response to the presence in the cell of another agent associated either with the pathogenic condition or with a specific cell type (an “identifying agent”). In addition, vector delivery may be controlled by targeting vector entry specifically to the desired cell type (for instance, a virally infected or malignant cell) as discussed above.

One method of administration is leukophoresis, in which about 20% of an individual's PBLs are removed at any one time and manipulated in vitro. Thus, approximately 2×10⁹ cells may be treated and replaced. Repeat treatments may also be performed. Alternatively, bone marrow may be treated and allowed to amplify the effect as described above. In addition, packaging cell lines producing a vector may be directly injected into a subject, allowing continuous production of recombinant virions.

In one embodiment, FIV vector particles which express RNA complementary to key pathogenic gene transcripts (for example, a viral gene product or an activated cellular oncogene) can be used to inhibit translation of that transcript into protein, such as the inhibition of translation of the HIV tat protein. Since expression of this protein is essential for viral replication, cells containing the FIV vector particle would be resistant to HIV replication.

In a second embodiment, where the pathogenic agent is a single-stranded virus having a packaging signal, RNA complementary to the viral packaging signal (e.g., an HIV packaging signal when the palliative is directed against HIV) is expressed, so that the association of these molecules with the viral packaging signal will, in the case of retroviruses, inhibit stem loop formation or tRNA primer binding required for proper encapsidation or replication.

In a third embodiment, FIV vector particles may be introduced which expresses a palliative capable of selectively inhibiting the expression of a pathogenic gene, or a palliative capable of inhibiting the activity of a protein produced by the pathogenic agent. In the case of HIV, one example is a mutant tat protein which lacks the ability to transactivate expression from the HIV LTR and interferes (in a transdominant manner) with the normal functioning of tat protein. Such a mutant has been identified for HTLV II tat protein (“XII Leu⁵” mutant; see Wachsman et al., Science 235:674, 1987). A mutant transrepressor tat should inhibit replication much as has been shown for an analogous mutant repressor in HSV-1 (Friedmann et al., Nature 335:452, 1988).

Such a transcriptional repressor protein can be selected for in tissue culture using any viral-specific transcriptional promoter whose expression is stimulated by a virus-specific transactivating protein (as described above). In the specific case of HIV, a cell line expressing HIV tat protein and the HSVTK gene driven by the HIV promoter will die in the presence of ACV. However, if a series of mutated tat genes are introduced to the system, a mutant with the appropriate properties (i.e., represses transcription from the HIV promoter in the presence of wild-type tat) will grow and be selected. The mutant gene can then be reisolated from these cells. A cell line containing multiple copies of the conditionally lethal vector/tat system may be used to assure that surviving cell clones are not caused by endogenous mutations in these genes. A battery of randomly mutagenized tat genes are then introduced into these cells using a “rescuable” FIV vector (i.e., one that expresses the mutant tat protein and contains a bacterial origin of replication and drug resistance marker for growth and selection in bacteria). This allows a large number of random mutations to be evaluated and permits facile subsequent molecular cloning of the desired mutant cell line. This procedure may be used to identify and utilize mutations in a variety of viral transcriptional activator/viral promoter systems for potential antiviral therapies.

b. Conditional Toxic Palliatives

Another approach for inhibiting a pathogenic agent is to express a palliative which is toxic for the cell expressing the pathogenic condition. In this case, expression of the palliative from the FIV vector should be limited by the presence of an entity associated with the pathogenic agent, such as a specific viral RNA sequence identifying the pathogenic state, in order to avoid destruction of nonpathogenic cells. In one embodiment of this method, FIV vector particles can be utilized to express a toxic gene (as discussed above) from a cell-specific responsive vector. In this manner, rapidly replicating cells, which contain the RNA sequences capable of activating the cell-specific responsive vectors, are preferentially destroyed by the cytotoxic agent produced by the FIV vector particle.

In a similar manner to the preceding embodiment, the FIV vector can carry a gene for phosphorylation, phosphoribosylation, ribosylation, or other metabolism of a purine- or pyrimidine-based drug. This gene may have no equivalent in mammalian cells and might come from organisms such as a virus, bacterium, fungus, or protozoan. An example of this would be the E. coli guanine phosphoribosyl transferase gene product, which is lethal in the presence of thioxanthine (see Besnard et al., Mol. Cell. Biol. 7:4139-4141, 1987). Conditionally lethal gene products of this type (also referred to as “pro-drugs” or “prodrug activating enzymes”) have application to many presently known purine- or pyrimidine-based anticancer drugs, which often require intracellular ribosylation or phosphorylation in order to become effective cytotoxic agents. The conditionally lethal gene product could also metabolize a nontoxic drug which is not a purine or pyrimidine analogue to a cytotoxic form (see Searle et al., Brit. J. Cancer 53:377-384, 1986).

In another aspect of the present invention, FIV vectors are provided which direct the expression of a gene product capable of activating an otherwise inactive precursor into an active inhibitor of the pathogenic agent. For example, the HSVTK gene product may be used to more effectively metabolize potentially antiviral nucleoside analogues such as AZT or ddC. The HSVTK gene may be expressed under the control of a cell-specific responsive vector and introduced into these cell types. AZT (and other nucleoside antivirals) must be metabolized by cellular mechanisms to the nucleotide triphosphate form in order to specifically inhibit retroviral reverse transcriptase, and thus, HIV replication (Furmam et al., Proc. Natl. Acad. Sci. USA 83:8333-8337, 1986). Constitutive expression of HSVTK (a nucleoside and nucleoside kinase with very broad substrate specificity) results in more effective metabolism of these drugs to their biologically active nucleotide triphosphate form. AZT or ddC therapy will thereby be more effective, allowing lower doses, less generalized toxicity, and higher potency against productive infection. Additional nucleoside analogues whose nucleotide triphosphate forms show selectivity for retroviral reverse transcriptase but, as a result of the substrate specificity of cellular nucleoside and nucleotide kinases are not phosphorylated, will be made more efficacious.

Administration of these FIV vector particles to human T cell and macrophage/monocyte cell lines can increase their resistance to HIV in the presence of AZT and ddC compared to the same cells without retroviral vector treatment. Treatment with AZT would be at lower than normal levels to avoid toxic side effects but still efficiently inhibit the spread of HIV. The course of treatment would be as described for the blocker.

In one embodiment, the FIV vector particle carries a gene specifying a product which is not in itself toxic but, when processed or modified by a protein such as a protease specific to a viral or other pathogen, is converted into a toxic form. For example, the FTV vector could carry a gene encoding a proprotein for ricin A chain, which becomes toxic upon processing by the HIV protease. More specifically, a synthetic inactive proprotein form of the toxin ricin or diphtheria A chains could be cleaved to the active form by arranging for the HIV virally encoded protease to recognize and cleave off an appropriate “pro” element.

In another embodiment, the FIV vector particle may express a “reporting product” on the surface of the target cells in response to the presence of an identifying agent in the cells (such as expression of a viral gene). This surface protein can be recognized by a cytotoxic agent, such as antibodies for the reporting protein, or by cytotoxic T cells. In a similar manner, such a system can be used as a detection system (see below) to simply identify those cells having a particular gene which expresses an identifying protein. Similarly, in another embodiment, a surface protein could be expressed which would itself be therapeutically beneficial. In the particular case of HIV, expression of the human CD4 protein specifically in HIV-infected cells may be beneficial in two ways:

1. Binding of CD4 to HIV env intracellularly could inhibit the formation of viable viral particles, much as soluble CD4 has been shown to do for free virus, but without the problem of systematic clearance and possible immunogenicity, since the protein will remain membrane bound and is structurally identical to endogenous CD4 (to which the patient should be immunologically tolerant).

2. Since the CD4/HIV env complex has been implicated as a cause of cell death, additional expression of CD4 (in the presence of excess HIV-env present in HIV-infected cells) leads to more rapid cell death and thus inhibits viral dissemination. This may be particularly applicable to monocytes and macrophages, which act as a reservoir for virus production as a result of their relative refractility to HIV-induced cytotoxicity (which, in turn, is apparently due to the relative lack of CD4 on their cell surfaces). In another embodiment, the FIV vector particle can provide a ribozyme which will cleave and inactivate RNA molecules essential for viability of the vector infected cell. By making ribozyme production dependent on a specific RNA sequence corresponding to the pathogenic state, such as HIV tat, toxicity is specific to the pathogenic state.

3. Expression of Markers

The above-described technique of expressing a palliative in a cell in response to a specific RNA sequence can also be modified to enable detection of a particular gene in a cell which expresses an identifying protein (for example, a gene carried by a particular virus), and hence enable detection of cells carrying that virus. In addition, this technique enables the detection of viruses (such as HIV) in a clinical sample of cells carrying an identifying protein associated with the virus.

This modification can be accomplished by providing a genome coding for a product, the presence of which can be readily identified (the “marker product”), in a FIV vector which responds to the presence of the identifying protein in the infected cells. For example, HIV, when it infects suitable cells, makes tat and rev. The indicator cells can thus be provided with a genome (such as by infection with an appropriate FIV virus particle) which codes for a marker gene, such as the alkaline phosphatase gene, b-galactosidase gene, or the luciferase gene which is expressed by the FIV particle upon activation by the tat and/or rev RNA transcript. In the case of β-galactosidase or alkaline phosphatase, exposing the cells to substrate analogues results in a color or fluorescence change if the sample is positive for HIV. In the case of luciferase, exposing the sample to luciferin will result in luminescence if the sample is positive for HIV. For intracellular enzymes such as β-galactosidase, the viral titer can be measured directly by counting colored or fluorescent cells, or by making cell extracts and performing a suitable assay. For the membrane bond form of alkaline phosphatase, virus titer can also be measured by performing enzyme assays on the cell surface using a fluorescent substrate. For secreted enzymes, such as an engineered form of alkaline phosphatase, small samples of culture supernatant are assayed for activity, allowing continuous monitoring of a single culture over time. Thus, different forms of this marker system can be used for different purposes. These include counting active virus, or sensitively and simply measuring viral spread in a culture and the inhibition of this spread by various drugs.

Further specificity can be incorporated into the preceding system by testing for the presence of the virus either with or without neutralizing antibodies to that virus. For example, in one portion of the clinical sample being tested, neutralizing antibodies to HIV may be present; whereas in another portion there would be no neutralizing antibodies. If the tests were negative in the system where there were antibodies and positive where there were no antibodies, this would assist in confirming the presence of HIV.

Within an analogous system for an in vitro assay, the presence of a particular gene, such as a viral gene, may be determined in a cell sample. In this case, the cells of the sample are infected with a suitable FIV vector particle which carries the reporter gene which is only expressed in the presence of the appropriate viral RNA transcript. The reporter gene, after entering the sample cells, will express its reporting product (such as b-galactosidase or luciferase) only if the host cell expresses the appropriate viral proteins. These assays are more rapid and sensitive, since the reporter gene can express a greater amount of reporting product than identifying agent present, which results in an amplification effect. 4. Immune Down-Regulation

As described above, the present invention also provides FIV vector particles capable of suppressing one or more elements of the immune system in target cells infected with the FIV vector particles. Briefly, specific down-regulation of inappropriate or unwanted immune responses, such as in chronic hepatitis or in transplants of heterologous tissue such as bone marrow, can be engineered using immune-suppressive viral gene products which suppress surface expression of transplantation (MHC) antigen. Group C adenoviruses Ad2 and Ad5 possess a 19 kd glycoprotein (gp 19) encoded in the E3 region of the virus. This gp 19 molecule binds to class I MHC molecules in the endoplasmic reticulum of cells, and prevents terminal glycosylation and translocation of class I MHC to the cell surface. For example, prior to bone marrow transplantation, donor bone marrow cells may be infected with a gp 19-encoding FIV vector which, upon expression of the gp 19, inhibit the surface expression of MHC class I transplantation antigens. These donor cells may be transplanted with low risk of graft rejection and may require a minimal immunosuppressive regimen for the transplant patient. This may allow an acceptable donor-recipient chimeric state to exist with fewer complications. Similar treatments may be used to treat the range of so-called autoimmune diseases, including lupus erythromiatis, multiple sclerosis, rheumatoid arthritis or chronic hepatitis B infection.

An alternative method involves the use of anti-sense message, ribozyme, or other specific gene expression inhibitor specific for T cell clones which are autoreactive in nature. These block the expression of the T cell receptor of particular unwanted clones responsible for an autoimmune response. The anti-sense, ribozyme, or other gene may be introduced using the FIV vector delivery system. 5. Replacement or Augmentation Gene Therapy

One further aspect of the present invention relates to transforming cells of a vertebrate or insect with a FIV vector which supplies genetic sequences capable of expressing a therapeutic protein. Within one embodiment of the present invention, the FIV vector is designed to express a therapeutic protein capable of preventing, inhibiting, stabilizing or reversing an inherited or noninherited genetic defect in metabolism, immune regulation, hormonal regulation, enzymatic or membrane associated structural function. This embodiment also describes the FIV vector particle capable of transducing individual cells, whereby the therapeutic protein is able to be expressed systemically or locally from a specific cell or tissue, whereby the therapeutic protein is capable of (a) the replacement of an absent or defective cellular protein or enzyme, or (b) supplement production of a defective of low expressed cellular protein or enzyme. Such diseases may include cystic fibrosis, Parkinson's disease, hypercholesterolemia, adenosine deaminase deficiency, β-globin disorders, Hemophilia A & B, Gaucher's disease, diabetes and leukemia. a. Treatment of Gaucher disease

As an example of the present invention, FIV vector particles can be constructed and utilized to treat Gaucher disease. Briefly, Gaucher disease is a genetic disorder that is characterized by the deficiency of the enzyme glucocerebrosidase. This type of therapy is an example of a single gene replacement therapy by providing a functional cellular enzyme. This enzyme deficiency leads to the accumulation of glucocerebroside in the lysosomes of all cells in the body. However, the disease phenotype is manifested only in the macrophages, except in the very rare neuronpathic forms of the disease. The disease usually leads to enlargement of the liver and spleen and lesions in the bones. (For a review, see Science 256:794, 1992, and The Metabolic Basis of Inherited Disease, 6th ed., Scriver et al., vol. 2, p. 1677). b. FIV vector particles Expressing Human Factor VIII and Factor IX for Treatment of Hemophilia

Within one embodiment of the invention, FIV vector particles expressing a B-domain deleted factor VIII protein are provided (see also PCT WO 91/09122, and Attorney's Docket No. 1155.005 entitled “Methods for Administration of Recombinant Gene Delivery Vehicles for Treatment of Hemophilia and Other Disorders”). Briefly, the B domain separates the second and third A domains of factor FVIII in the newly synthesized single-chain molecule. The B domain extends from amino acids 712 to 1648 according to Wood et al., 1984, Nature 312:330-337. Proteolytic activation of factor VIIII involves cleavage at specific Arg residues located at positions 372, 740, and 1689. Cleavages of plasma factor VIII by thrombin or Factor Xa at Arg 372 and Arg 1689 are essential for factor VIII to participate in coagulation. Therefore, activated factor VIII consists of a heterodimer comprising amino acids residues 1-372 (containing the Al domain) and residues 373-740 (containing the A2 domain), and residues 1690-2332 (containing the A3-C1-C2 domain).

An important advantage in using the B domain deleted FVIII molecule is that the reduced size appears to be less prone to proteolytic degradation and therefore, no addition of plasma-derived albumin is necessary for stabilization of the final product. The term “B domain deletion” as used herein with respect to factor VIII protein refers to a factor VIII protein in which some or all removal of some or all of the amino acids between residues 711 and 1694 have been deleted, and which still preserves a biologically active FVIII molecule.

A range of B domain deletions can exist depending on which amino acid residues in the B domain is deleted and whereby the biological activity of the FVIII molecule is still preserved. A specific B domain deletion called the SQN exists which is created by fusing Ser 743 to Gln 1638 (Lind et al., 1995, Eur J. Biochem 323:19-27, and PCT WO 91/09122) This deletes amino acid residues 744 to 1637 from the B domain creating a Ser-Glu-Asn (SQN) link between the A2 and A3 FVIII domains. When compared to plasma-derived FVIII, the SQN deletion of the B domain of FVIII did not influence its in vivo pharmacokinetics (Fijnvandraat, et. al., P. R. Schattauer Vertagsgesellschatt mbH (Stuttgart) 77:298-302, 1997). The terms “Factor VIII SQN deletion” or “SQN deletion” as used herein refer to this deletion and to other deletions which preserve the single S-Q-N tripeptide sequence and which result in the deletion of the amino acids between the two B-domain SQN sequences (See PCT WO 91/09122 for a description of this amino acid sequence).

There are number of other B-domain deleted forms of factor VIII. cDNA's encoding all of these B-domain deleted factor VIII proteins can be inserted into FIV vector particles by using standard molecular biology techniques. For example cDNA molecules encoding the following B-domain factor VIII deletions can be constructed as described below:

Eaton (1986) Biochemistry 25:8343des 797-1562 deletionToole (1986) PNAS 83:5939des 760-1639 (LA-FVIII) Meutien (1988) Prot Eng 2:301des 771-1666 (FVIII del II: missing one thrombin site) Sarver (1987) DNA 6:553des 747-1560Mertens (1993) Br J Haematol 85:133des 868-1562

des 713-1637 (thrombin resistant) Esmon (1990) Blood 76:1593des 797-1562Donath (1995) Biochem J 312:49des 741-1668Webb (1993) BBRC 190:536PCR cloned from mRNALind (1995) Eur J Biochem 232:19des 748-1648 (partially processed)

des 753-1648(partially processed)

des 777-1648(partially processed)

des 744-1637 (FVIII-SQ)

des 748-1645 (FVIII-RH)

des B-domain +0, 1 ,2 Arg (partially processed)

desB, +3Arg (FVIIIR4)

desB, +4Arg (FVIIIR5) Langner (1988) Behring Inst Mitt 16-25des 741-1689

des 816-1598Cheung (1996) Blood 88:325ades 746-1639Pipes (1996) Blood 88:441ades 795-1688 (thrombin sites mutated)

A B domain deletion in which an IgG hinge region has been inserted can also be used. For instance, a deletion of this type can be obtained from plasmid pSVF8-tb2, which was designed to link the heavy and light chains with a short hinge region from immunoglobulin A. To obtain cleavage at the end of the heavy chain and to release the light chain, some residues of the b domain are included on either side of the hinge sequence. The 5′ untranslated leader and signal peptide are from the human Factor VIII:C cDNA, with the Kozak consensus sequence at the initiation codon as in pSVF8-302. A description of this vector is included in Chapman et al., U.S. Pat. No. 5,595,886. The 3′ untranslated region is the same fused Factor VIII and tPA sequence as found in pSVF8-80K.

The construction may be completed in two steps: an oligomer with cohesive ends for EcoRI and BclI (117 bp) wa cloned into a transfer vector, pF8GM7, the DNA sequence of the oligomer was checked by ml 3 subcloning and Sanger sequencing. Next, the final plasmid was assembled by ligation of the following three fragments:

(a) FspI-EcoRI fragment form pSVF8-92S;

(b) EcoRI-NdeI fragment of the transfer vector pF8GM7 with oligomer; and

(c) FspI-NdeI fragment of pSVF8-80K.

Descriptions of pSVF8-92S and pSVF8-80K are included in Chapman et al., U.S. Pat. No. 5,595,886.

Three additional B domain-deleted factor VIII constructs of particular interest for inclusion in the FIV vector particles of the invention can be prepared as follows. Plasmid pSVF8-500 encodes a factor VIII protein with amino acids 770 to 1656 of the full length Factor VIII deleted. In addition the threonine at position 1672 of the full-length factor VIII sequence was also deleted. The following is a description of the construction of the vector.

The pSVF8-500 plasmid is a derivative of pSVF8-302 in which the regions coding for the 92K and 80K domains are fused with a small connecting b-region of 21 amino acids, retaining the natural proteolytic processing sites. This plasmid was constructed in the following manner:

(1) A SalI-KpnI fragment of 1984 bp containing the region coding for the 92K protein (except for the carboxyl terminal end) and BstXI-SalI fragment of 2186 bp containing the region coding for the carboxyl end of the 80K protein with 3′ end untranslated region were isolated by gel electrophoresis after digestion of pSVF8-302 with restriction enzymes. (2) A BclI-BstXI fragment of 1705 bp containing most of the region coding for the 80K protein was isolated after gel electrophoresis of the BamHI-XbaI fragment of pUC12F8. (pUCF812 is prepared from pF8-102 which is described in U.S. Pat. No. 5,045,455. pF8-102 is digested with Bam-XhaI and ligated into vector pUC12 by in vitro mutagenesis at a BclI site using the following primer: 5′ ACT ACT CTT CAA TCT GAT CAA GAG GAA 3′ (Seq ID No. ______).

(3) A KpnI-EcoRI fragment containing the carboxyl end of the 92K protein and part of the b region (4 amino acids) was obtained by digestion of the SalI cassette from pSVF8-302 with KpnI and EcoRI.

(4) Ligation of four pieces of synthetic DNA to the fragments of steps (2) and (3) and digestion with KpnI.

(5) Final ligation of fragments from steps (1) and (4); digestion with SalI and gel purification of the 6428 bp SalI cassette.

(6) Ligation of the SalI cassette into pSV7d vector; transformation of HB101 and colony hybridization to isolate pSVF8-500. The sequence of the junction region coding for 92K-b-80K was verified by DNA sequence after cloning in M13. The sequence was changed to incorporate unique NruI and MluI restriction sites without changing the amino acid sequence. These sites were alsoused to construct other two additional B-domain deleted vectors which are described below.

pSV500BDThr was constructed from pSVF8-500. The threonine deletion at position 1672 was maintained. A synthetic linker was used to construct pSV500BDThr. The linker extends from a unique NruI site at Ser(765) to a unique MluI site at Ile(1659) in the pSVF8-500 vector. This linker was substituted for the corresponding region of pSVF8-500.

A third vector pSVF8-500B was constructed from pSV500BDThr. This vector is identical to pSVF8-500B except that the codon for threonine 1672 was re-inserted using standard mutagenesis methods. The relationship between, pSVF8-500B, pSVF8-500B, is further illustrated in the table below. Amino acid sequence numbers in the table were determined by reference to full-length factor VIII sequence.

In all cases, the BglII-PflI 1.35 kb fragments of each modified cDNA listed above can be inserted into the FIV vector particles described herein using standard molecular biology procedures known to those of skill in the art and described herein.

The full-length factor VIII cDNA can also be inserted into the FIV vector particles of the invention (see, e.g., WO 96/21035). A variety of Factor VIII deletions, mutations, and polypeptide analogs of Factor VIII can also be introduced into the FIV vector particles of the invention including FIV vector particles by modifications of the procedures described herein. These analogs include, for instance, those described in PCT Patent Publication Nos. WO 97/03193, WO 97/03194, WO 97/03195, and WO 97/03191, all of which are hereby incorporated by reference.

Hemophilia B can also be treated with systemically administered factor IX-expressing FIV vector particles including FIV vector particles. Human factor IX deficiency (Christmas disease or Hemophilia B) affects primarily males because it is transmitted as sex-linked recessive trait. It affects about 2000 people in the US. The human factor gene codes a 416 amino acids of mature protein.

The human factor IX cDNA can be obtained for instance by constructing plasmid pHfIX1, as described by Kurachi and Davie, 1982, PNAS 79(21):6461-6464. The cDNA sequence can be excised as a PstI fragment of about 1.5 kb, blunt ended using T4 DNA polymerase. The factor cDNA fragment can be readily inserted, for example into a SrfI site introduced into a FIV vector particle.

c. FIV1 Vector Particles Expressing Other Clotting Factors

i Factor V.

FIV vector particles can be constructed using molecular biology techniques known to those of skill in the art. For instance, Factor V cDNA is obtained from pMT2-V (Jenny, 1987, Proc. Natl. Acad. Sci. USA 84:4846; ATCC deposit #40515) by digestion with SalI. The 7 kb cDNA band is excised from agarose gels and cloned into FIV vector particles, using standard molecular biology techniques.

Either a full-length or a B-domain deletion or substitution of the factor V cDNA can be expressed by the gene therapy vectors of the invention. Factor V B-domain deletions such as those reported by Marquette, 1995, Blood 86:3026, and Kane, 1990, Biochemistry 29:6762, can be made as described by these authors.

ii. Antithrombin III

FIV vector particles capable of expressing ATIII cDNA can be readily constructed using standard molecular biology techniques known to those of skill in the art. For instance a FIV vector particle expressing AT III can be constructed from the vector pKT218 (Prochownik, 1983, J. Biol. Chem. 258:8389; ATCC number 57224/57225) by excision with PstI. The 1.6 kb cDNA insert can be recovered from agarose gels and cloned into the PstI site of vector SK-. The insert can be recovered by restriction enzyme digestion and cloned into FIV vector particles described herein by the restriction enzymes.

iii. Protein C

The FIV vector particles of the invention capable of expressing Protein C can be made using a wide variety of techniques given the present disclosure. For instance, protein C cDNA will be obtained by restriction enzyme digestion of published vector (Foster, 1984, Proc. Natl. Acad. Sci. USA 81:4766; Beckmann, 1985, Nucleic Acids Res 13:5233). The 1.6 kb cDNA insert can be recovered from agarose gels and cloned into the multiple cloning site of vector SK− under standard conditions. The insert can be recovered by restriction enzyme digestion and cloned into a FIV vector; for example, excision by XhoI/NotI digestion followed by cloning into XhoI/NotI digested FIV vector.

iv. Prothrombin

FIV vector particles expressing prothrombin and its variants can be constructed by methods known to those of skill in the art, by using variations on the methods described herein. For instance, prothrombin cDNA can be obtained by restriction enzyme digestion of a published vector (Degen (1983) Biochemistry 22:2087). The 1.9 kb cDNA insert can be recovered from agarose gels and cloned into the multiple cloning site of vector SK-. The insert can be recovered by restriction enzyme digestion and cloned into a FIV vector using restriction enzyme digestion

v. Thrombomodulin

FIV vector particles expressing thrombomodulin and its variants can be constructed using techniques known to those of skill in the art. For instance, thrombomodulin cDNA can be obtained from the vector puc19TM15 (Jackman, 1987, Proc. Natl. Acad. Sci. USA 84:6425; Shirai, 1988, J. Biochem. 103:281; Wen, 1987, Biochemistry 26:4350; Suzuki, 1987, EMBO J 6:1891; ATCC number 61348,61349) by excision with SalI. The 3.7 kb cDNA insert can be recovered from agarose gels and cloned into the SalI site of lentiviral vector.

d. FIV Vector Particles Treatment of Hereditary Disorders and Other Conditions

There are a number of proteins useful for treatment of hereditary disorders that can be expressed in vivo by the methods of invention. Many genetic diseases caused by inheritance of defective genes result in the failure to produce normal gene products, for example, thalassemia, phenylketonuria, Lesch-Nyhan syndrome, severe combined immunodeficiency (SCID), hemophilia, A and B, cystic fibrosis, Duchenne's Muscular Dystrophy, inherited emphysema and familial hypercholesterolemia (Mulligan et al., 1993, Science 260:926; Anderson et al., 1992, Science 256:808; Friedman et al., 1989, Science 244:1275). Although genetic diseases may result in the absence of a gene product, endocrine disorders, such as diabetes and hypopituitarism, are caused by the inability of the gene to produce adequate levels of the appropriate hormone insulin and human growth hormone respectively.

Gene therapy by the methods of the invention is a powerful approach for treating these types of disorders. This therapy involves the introduction of normal recombinant genes into somatic cells so that new or missing proteins are produced inside the cells of a patient. A number of genetic diseases can be treated by gene therapy, including adenine deaminase deficiency, cystic fibrosis, a₁-antitrypsin deficiency, Gaucher's syndrome, as well as non-genetic diseases. Other representative diseases include lactase for treatment of hereditary lactose intolerance, AD for treatment of ADA deficiency, and alpha-1 antitypsin for treatment of alpha-i antitrypsin deficiency. See F. D. Ledley, 1987, J. Pediatics 110:157-174; I. Verma, Scientific American (Nov., 1987) pp. 68-84; and PCT Patent Publication WO 95/27512 entitled “Gene Therapy Treatment for a Variety of Diseases and Disorders” for a description of gene therapy treatment of genetic diseases.

One such disorder is familial hypercholesterolemia is a disease characterized clinically by a lifelong elevation of low density lipoprotein (LDL), the major cholesterol-transport lipoprotein in human plasma; Pathologically by the deposition of LDL-derived cholesterol in tendons, skin and arteries leading to premnature coronary heart disease; and genetically by autosomal dominant inherited trait. Heterozygotes number about 1 in 500 persons worldwide. Their cells are able to bind cholesterol at about half the rate of normal cells. Their plasma cholesterol levels show two fold elevation starting at birth. Homozygotes number 1 in 1 million persons They have severe cholesterolemia with death occurring usually before age 20. The disease (Arteriosclerosis) depends on geography. It affects 15.5 per 100,000 individuals in the U.S. (20,000 total) and 3.3 per 100,000 individuals in Japan. FIV vector particles expressing the LDL receptor for treatment of disorders manifesting with elevated serum LDL can be constructed by techniques known to those of skill in the art.

There are a variety of other proteins of therapeutic interest that can be expressed in vivo by FIV vector particles using the methods of the invention. For instance sustained in vivo expression of tissue factor inhibitory protein (TFPI) is useful for treatment of conditions including sepsis and DIC and in preventing reperfusion injury. (See PCT Patent Publications Nos. WO 93/24143,WO 93/25230 and WO 96/06637. Nucleic acid sequences encoding various forms of TFPI can be obtained, for example, as described in U.S. Pat. Nos. 4,966,852; 5,106,833; and 5,466,783, and can be incorporated in FIV vector as is described herein.

Other proteins of therapeutic interest such as erythropoietin (EPO) and leptin can also be expressed in vivo by FIV vector particles according to the methods of the invention. For instance EPO is useful in gene therapy treatment of a variety of disorders including anemia (see PCT publication number WO 95/13376 entitled “Gene Therapy for Treatment of Anemia”.) Sustained gene therapy delivery of leptin by the methods of the invention is useful in treatment of obesity. (See WO 96/05309 entitled “Obesity Polypeptides able to modulate body weight” for a description of the leptin gene and its use in the treatment of obesity. FIV vector particle expressing EPO or leptin can readily be produced using the methods described herein and the constructs described in these two patent publications.

A variety of other disorders can also be treated by the methods of the invention. For example, sustained in vivo systemic production of apolipoprotein E or apolipoprotein A by the FIV vector particles of the invention can be used for treatment of hyperlipidemia. (See Breslow, J. et al. Biotechnology 12, 365 (1994).) In addition, sustained production of angiotensin receptor inhibitor (T. L. Goodfriend, et al., 1996, N. Engl. J. Med. 334:1469) can effected by the gene therapy methods described herein. As yet an additional example, the long term in vivo systemic production of angiostatin by the lentiviral vector particles of the invention is useful in the treatment of a variety of tumors. (See O'Reilly et al., 1996, Nature Med. 2:689.

7. Lymphokines and Lymphokine Receptors

As noted above, the present invention also provides FIV vector particles which can, among other functions, direct the expression of one or more cytokines or cytokine receptors. Briefly, in addition to their role as cancer therapeutics, cytokines can have negative effects resulting in certain pathological conditions. For example, most resting T-cells, B cells, large granular lymphocytes and monocytes do not express IL-2R (receptor). In contrast to the lack of IL-2R expression on normal resting cells, IL-2R is expressed by abnormal cells in patients with certain leukemias (ATL, Hairy-cell, Hodgkins, acute and clronic granulocytic), autoimmune diseases, and is associated with allograft rejection. Interestingly, in most of these patients the serum concentration of a soluble form of IL-2R is elevated. Therefore, with certain embodiments of the invention therapy may be effected by increasing the serum concentration of the soluble form of the cytokine receptor. For example, in the case of IL-2R, a FIV vector can be engineered to produce both soluble IL-2R and IL-2R, creating a high affinity soluble receptor. In this configuration, serum IL-2 levels would decrease, inhibiting the paracrine loop. This same strategy also may be effective against autoimmune diseases. In particular, because some autoimmune diseases (e.g., Rheumatoid arthritis, SLE) also are associated with abnormal expression of IL-2, blocking the action of IL-2 by increasing the serum level of receptor may also be utilized in order to treat such autoimmune diseases.

In other cases inhibiting the levels of IL-1 may be beneficial. Briefly, IL-1 consists of two polypeptides, IL-1 and IL-1, each of which has pleiotropic effects. IL-1 is primarily synthesized by mononuclear phagocytes, in response to stimulation by microbial products or inflammation. There is a naturally occurring antagonist of the IL-1R, referred to as the IL-1 Receptor antagonist (“IL-1Ra”). This IL-1R antagonist has the same molecular size as mature IL-1 and is structurally related to it. However, binding of IL-1Ra to the IL-1R does not initiate any receptor signaling. Thus, this molecule has a different mechanism of action than a soluble receptor, which complexes with the cytokine and thus prevents interaction with the receptor. IL-1 does not seem to play an important role in normal homeostasis. In animals, antibodies to IL-1 receptors reduce inflammation and anorexia due to endotoxins and other inflammation inducing agents.

In the case of septic shock, IL-1 induces secondary compounds which are potent vasodilators. In animals, exogenously supplied IL-1 decreases mean arterial pressure and induces leukopenia. Neutralizing antibody to IL-1 reduced endotoxin-induced fever in animals. In a study of patients with septic shock who were treated with a constant infusion of IL-1R for three days, the 28 day mortality was 16% compared to 44% in patients who received placebo infusions. In the case of autoimmune disease, reducing the activity of IL-1 reduces inflammation. Similarly, blocking the activity of IL-1 with recombinant receptors can result in increased allograft survival in animals, again presumably by decreasing inflammation.

These diseases provide further examples where FIV vector particles may be engineered to produce a soluble receptor or more specifically the IL-1Ra molecule. For example, in patients undergoing septic shock, a single injection of IL-1Ra producing vector particles could replace the current approach requiring a constant infusion of recombinant IL-1R.

Cytokine responses, or more specifically, incorrect cytokine responses may also be involved in the failure to control or resolve infectious diseases. Perhaps the best studied example is non-healing forms of leishmaniasis in mice and humans which have strong, but counterproductive T_{H}2-dominated responses. Similarly, lepromotomatous leprosy is associated with a dominant, but inappropriate T_{H}2 response. In these conditions, FIV vector particles may be useful for increasing circulating levels of IFN gamma, as opposed to the site-directed approach proposed for solid tumor therapy. IFN gamma is produced by T_{H}-1 T-cells, and functions as a negative regulator of T_{H}-² subtype proliferation. IFN gamma also antagonizes many of the IL-4 mediated effects on B-cells, including isotype switching to IgE.

IgE, mast cells and eosinophils are involved in mediating allergic reaction. IL-4 acts on differentiating T-cells to stimulate T_{H}-2 development, while inhibiting T_{H}-1 responses. Thus, FIV-based gene therapy may also be accomplished in conjunction with traditional allergy therapeutics. One possibility is to deliver FIV vector particles which produces IL4R with small amounts of the offending allergen (i.e., traditional allergy shots). Soluble IL-4R would prevent the activity of IL-4, and thus prevent the induction of a strong T_{H}-² response.

a. FIV Vector Particles for Treatment of Viral Hepatitis

The FIV vector particles including FIV vectors and the methods of administration described are useful for treatment of viral hepatitis, including hepatitis B and hepatitis C. For instance, the FIV vector particles of the invention can be used to express interferon-alpha for treatment of viral hepatitis. While not wishing to be bound by theory, FIV vector particles injected intravenously preferentially transduce liver cells. Thus, the methods of intravenous delivery described herein for FIV vector particles can be used for treatment of liver diseases such as hepatitis and in particular viral hepatitis, in which therapeutic proteins expressed by the FIV vector particles can be delivered preferentially to the liver.

Currently, the only approved treatment for chronic hepatitis B, C and D infections is the use of alpha interferon 2a and 2b.Alpha-interferon is a secreted protein induced in B lymphocytes, macrophages and null lymphocytes by foreign cells, virus-infected cells, tumor cells, bacterial cells and products and viral envelopes. The mechanism of antiviral action of interferon is by inducing the synthesis of effector proteins: two of the most important are 2′,5′-oligo-adenylate synthetase (OAS) and dsRNA-dependent protein kinase (RDPK). OAS synthesizes adenylate oligomers that activate RNAaseL, which degrades viral single stranded RNA. RDPK phosphorylates initiation factor eIF-2a which results in the inhibition of viral protein translation. In addition to the direct antiviral effect, alpha interferon has immunomodulatory effects that are important against viral infections. These immunomodulatory effects are: enhancement of the expression of both Class I and class II major histocompatibility complex (MHC) molecules, modulation of the expression of the interleukin-2 receptor, TNF-a receptor, transferrin receptor, enhancement of spontaneous natural killer (NK) cell cytotoxicity and modulation of antibody production by B cells. In chronic hepatitis B infection, the beneficial effect of interferon alpha appears to be from the immunomodulatory effects, while in chronic hepatitis C infection, the beneficial effect is dependent on its antiviral activity. (Bresters, D., in Hepatitis C Virus, pp121-136, Reesink H W (ed), 1994). The mechanism of action in interferon alpha for treatment of chronic hepatitis D is poorly understood (Rizzetto, M. and Rosina, F. in Viral Hepatitis, pp. 363-369, Zuckerman, A. J. and Thomas H. C. (ed), 1993).

Localized expression of interferon alpha in the liver from a FIV vector particle can be an effective treatment for hepatitis. While not wishing to bound by theory, delivery of alpha interferon at the site of infection by the gene therapy vectors of the invention, including FIV vector particles, results in high local concentration of the cytokine thereby focusing the antiviral and immunological effects to the adjacent infected hepatocytes. A further advantage of this treatment is that the current systemic mode of systemic alpha interferon therapy may either be unnecessary or be reduced in dose and frequency of treatment. This reduction can reduce the adverse side effects associated with the systemic delivery of alpha interferon. Thus, the gene therapy approaches described herein may be used in combination with administration of alpha-interferon protein formulations.

The construction of a number of different FIV vector particles expressing interferon-alpha can be readily accomplished given the disclosure provided herein. There are at least 24 different human alpha interferon genes or pseudogenes. There are two distinct families (I and II); mature human alpha interferon (I) are 166 amino acids long (one is 165 amino acids ) whereas alpha interferon (II) have 172 amino acids. Eighteen genes are in the alpha interferon I family, including at least four pseudogenes. Six genes are in the alpha interferon II family, including five pseudogenes (Callard, R., and Gearing, A., Cytokine Facts Book, Academic Press, 1994 pp. 148-154). In Example 33 herein, we use alpha interferon 2a, 2b, 2c, 54 and 76, all members of the alpha interferon (I) family. Similar techniques can be used for inserting other members of the alpha interferon I family (such as alpha interferon F and N) into lentiviral vector particles. Thus other biologically active forms of alpha-interferon in addition to 2a, 2b, 2c, 54 and 76 as described herein can also be expressed by the FIV vector particles of the invention and used for treatment of viral hepatitis.

Patients with viral hepatitis can be treated a combination gene therapy approach. A FIV vector particle expressing a protein drug such as alpha-interferon can be administered intravenously or directly to the liver by methods described herein. This therapeutic approach can be combined with intramusuclar delivery of a FIV vector particle expressing a hepatitis B or hepatitis C antigen for inducing a immune response against the hepatitis virus. Specific hepatitis B and C antigens useful in this type of therapy and the construction of FIV vector particles expressing such antigens are described herein and in PCT Patent Publication No. WO 93/15207. In addition, molecularly cloned genomes which encode the hepatitis B virus may be obtained from a variety of sources including, for example, the American Type Culture Collection (ATCC, Rockville, Md.). For example, ATCC No. 45020 contains the total genomic DNA of hepatitis B (extracted from purified Dane particles) (see FIG. 3 of Blum et al., 1989, TIG 5(5):154-158) in the Bam HI site of pBR322 (Moriarty et al., 1981, Proc. Natl. Acad. Sci. USA 78:2606-2610). (Note that correctable errors occur in the sequence of ATCC No. 45020.)

8. Suicide Vectors

One further aspect of the present invention relates to the use of FIV suicide vectors to limit the spread of wild-type lentivirus in the packaging/producer cell lines. For example, within one embodiment the FIV vector particles contains a prodrug activating enzyme as discussed above which, upon administration of the prodrug (e.g., gancyclovir) results in the death of cells containing the vector particles.

9. FIV Vectors to Prevent the Spread of Metastatic Tumors

One further aspect of the present invention relates to the use of FIV vector particles for inhibiting or reducing the invasiveness of malignant neoplasms. Briefly, the extent of malignancy typically relates to vascularization of the tumor. One cause for tumor vascularization is the production of soluble tumor angiogenesis factors (TAF) (Paweletz et al., Crit. Rev. Oncol. Hematol. 9:197, 1989) expressed by some tumors. Within one aspect of the present invention, tumor vascularization may be slowed utilizing FIV vectors to express antisense or ribozyme RNA molecules specific for TAF. Alternatively, anti-angiogenesis factors (Moses et al., Science 248:1408, 1990; Shapiro et al., PNAS 84:2238, 1987) may be expressed either alone or in combination with the above-described ribozymes or antisense sequences in order to slow or inhibit tumor vascularization. Alternatively, FIV vector particles can also be used to express an antibody specific for the TAF receptors on surrounding tissues.

10. Modulation of Transcription Factor Activity

In yet another embodiment, FIV vector particles may be utilized in order to regulate the growth control activity of transcription factors in the infected cell. Briefly, transcription factors directly influence the pattern of gene expression through sequence-specific trans-activation or repression (Karin, New Biologist 21:126-131, 1990). Thus, it is not surprising that mutated transcription factors represent a family of oncogenes. FIV vector particles can be used, for example, to return control to tumor cells whose unregulated growth is activated by oncogenic transcription factors, and proteins which promote or inhibit the binding cooperatively in the formation of homo- and heterodimer trans-activating or repressing transcription factor complexes.

One method for reversing cell proliferation would be to inhibit the trans-activating potential of the c-myc/Max heterodimer transcription factor complex. Briefly, the nuclear oncogene c-myc is expressed by proliferating cells and can be activated by several distinct mechanisms, including retroviral insertion, amplification, and chromosomal translocation. The Max protein is expressed in quiescent cells and, independently of c-myc, either alone or in conjunction with an unidentified factor, functions to repress expression of the same genes activated by the myc/Max heterodimer (Cole, Cell 65:715-716, 1991).

Inhibition of c-myc or c-myc/Max proliferation of tumor cells may be accomplished by the overexpression of Max in target cells controlled by FIV vectors. The Max protein is only 160 amino acids (corresponding to 480 nucleotide RNA length) and is easily incorporated into a FIV vector either independently, or in combination with other genes and/or antisense/ribozyme moieties targeted to factors which release growth control of the cell.

Modulation of homo/hetero-complex association is another approach to control transcription factor activated gene expression. For example, transport from the cytoplasm to the nucleus of the trans-activating transcription factor NF-B is prevented while in a heterodimer complex with the inhibitor protein IB. Upon induction by a variety of agents, including certain cytokines, IB becomes phosphorylated and NF-B is released and transported to the nucleus, where it can exert its sequence-specific trans-activating function (Baeuerle and Baltimore, Science 242:540-546, 1988). The dissociation of the NF-B/IB complex can be prevented by masking with an antibody the phosphorylation site of TB. This approach would effectively inhibit the trans-activation activity of the NF-IB transcription factor by preventing its transport to the nucleus. Expression of the IB phosphorylation site specific antibody or protein in target cells may be accomplished with a FIV gene transfer vector. An approach similar to the one described here could be used to prevent the formation of the trans-activating transcription heterodimer factor AP-1 (Turner and Tijan, Science 243:1689-1694, 1989), by inhibiting the association between the jun and fos proteins.

11. FIV Vector Particle Delivery to Cats

In one embodiment of the present invention, FIV vector particles are used to deliver heterologous genes to cats. Gene delivery to cats using the cat-specific delivery system based on FIV can be used for various purposes and establishes and small animal model where many applications and parameters of gene delivery can be easily studied in an in vivo situation.

Within one aspect of the invention, FIV vector particles are used for veterinary applications by introducing heterologous genes to cats in order to vaccinate for various feline diseases and/or deliver therapeutic genes to improve the health for genetic disorders, cancers or viral diseases of cats. The efficiency and level of gene expression in cats is expected to be very high since the heterologous gene is driven by the FIV LTR. Therefore, this gene delivery approach might have an advantage over existing methods of vaccination and/or introduction of heterologous genes into cats.

Within another aspect of the invention, marking and repopulation studies are carried out in a cat model after transduction of hematopoietic cells.

Furthermore, the implications of certain heterologous genes that might help fight FIV disease (e.g. antisense DNA sequences, cytokines) are introduced with the FIV vector particles and studied in the feline system. The FIV disease progression in cats is very similar to the HIV disease progression in humans. This small animal model might therefore give valuable insight in possible treatments of HIV. Furthermore, the effectiveness of various attenuated FIV viruses can easily be studied in cats and might lead to the development of attenuated HIV viruses that effectively protect the host to new wildtype virus challenge.

Within another aspect, FIV vector particles are used to deliver genes to feline dendritic cells. Using this cat model, an in vivo comparative study of the potential to present antigen and elicit efficacious immune responses of dendritic cells versus other APCs can be examined. Theses studies might give valuable insight into the function of the immune system and allow an analysis of various parameters of gene delivery (e.g. type of antigen, dose, route of delivery, time course) in an in vivo situation.

Formulation

Within other aspects of the present invention, methods are provided for preserving an infectious FIV vector particle, such that the FIV vector particle is capable of infecting mammalian cells upon reconstitution (see U.S. Ser. No. 08/153,342). Briefly, FIV vector particles which have been purified or concentrated may be preserved by first adding a sufficient amount of a formulation buffer to the media containing the FIV vector particles, in order to form an aqueous suspension. The formulation buffer is an aqueous solution that contains a saccharide, a high molecular weight structural additive, and a buffering component in water. As utilized within the context of the present invention, a “buffering compound” or “buffering component” should be understood to refer to a substance that functions to maintain the aqueous suspension at a desired pH. The aqueous solution may also contain one or more amino acids.

The FIV vector particle can also be preserved in a purified form. More specifically, prior to the addition of the formulation buffer, the crude FIV vector particle described above may be clarified by passing it through a filter, and then concentrated, such as by a cross flow concentrating system (Filtron Technology Corp., Nortborough, Mass.). Within one embodiment, DNase is added to the concentrate to digest exogenous DNA. The digest is then diafiltrated to remove excess media components and establish the FIV vector particle in a more desirable buffered solution. The diafiltrate is then passed over a Sephadex S-500 gel column and a purified FIV vector particle is eluted. A sufficient amount of formulation buffer is added to this eluate to reach a desired final concentration of the constituents and to minimally dilute the FIV vector particle, and the aqueous suspension is then stored, preferably at −70° C. or immediately dried. As noted above, the formulation buffer is an aqueous solution that contains a saccharide, a high molecular weight structural additive, and a buffering component in water. The aqueous solution may also contain one or more amino acids.

The crude FIV vector particle can also be purified by ion exchange column chromatography (see U.S. patent application Ser. No. 08/093,436). In general, the crude FIV vector particle is clarified by passing it through a filter, and the filtrate loaded onto a column containing a highly sulfonated cellulose matrix. The FIV vector particle is eluted from the column in purified form by using a high salt buffer. The high salt buffer is then exchanged for a more desirable buffer by passing the eluate over a molecular exclusion column. A sufficient amount of formulation buffer is then added, as discussed above, to the purified FIV vector particle and the aqueous suspension is either dried immediately or stored, preferably at −70° C.

The aqueous suspension in crude or purified form can be dried by lyophilization or evaporation at ambient temperature. Specifically, lyophilization involves the steps of cooling the aqueous suspension below the glass transition temperature or below the eutectic point temperature of the aqueous suspension, and removing water from the cooled suspension by sublimation to form a lyophilized lentivirus. Briefly, aliquots of the formulated FIV vector particle are placed into an Edwards Refrigerated Chamber (3 shelf RC3S unit) attached to a freeze dryer (Supermodulyo 12K). A multistep freeze drying procedure as described by Phillips et al. (Cryobiology 18:414, 1981) is used to lyophilize the formulated FIV vector particle, preferably from a temperature of −40° C. to −45° C. The resulting composition contains less than 10% water by weight of the lyophilized lentivirus. Once lyophilized, the FIV vector particle is stable and may be stored at −20° C. to 25° C. Within the evaporative method, water is removed from the aqueous suspension at ambient temperature by evaporation. Within one embodiment, water is removed through spray drying (EP 520,748). Within the spray drying process, the aqueous suspension is delivered into a flow of preheated gas, usually air, whereupon water rapidly evaporates from droplets of the suspension. Spray drying apparatus are available from a number of manufacturers (e.g., Drytec, Ltd., Tonbridge, England; Lab-Plant, Ltd., Huddersfield, England). Once dehydrated, the FIV vector particle is stable and may be stored at −20° C. to 25° C. Within the methods described herein, the resulting moisture content of the dried or lyophilized lentivirus may be determined through use of a Karl-Fischer apparatus (EM Science Aquastar' VIB volumetric titrator, Cherry Hill, N.J.), or through a gravimetric method. The aqueous solutions used for formulation, as previously described, are composed of a saccharide, high molecular weight structural additive, a buffering component, and water. The solution may also include one or more amino acids. The combination of these components act to preserve the activity of the FIV vector particle upon freezing and lyophilization, or drying through evaporation. Although a preferred saccharide is lactose, other saccharides may be used, such as sucrose, mannitol, glucose, trehalose, inositol, fructose, maltose or galactose. In addition, combinations of saccharides can be used, for example, lactose and mannitol, or sucrose and mannitol (e.g., a concentration of lactose is 3%-4% by weight. Preferably, the concentration of the saccharide ranges from 1% to 12% by weight.

The high molecular weight structural additive aids in preventing viral aggregation during freezing and provides structural support in the lyophilized or dried state. Within the context of the present invention, structural additives are considered to be of “high molecular weight” if they are greater than 5000 m.w. A preferred high molecular weight structural additive is human serum albumin. However, other substances may also be used, such as hydroxyethyl-cellulose, hydroxymethyl-cellulose, dextran, cellulose, gelatin, or povidone. A particularly preferred concentration of human serum albumin is 0.1% by weight. Preferably, the concentration of the high molecular weight structural additive ranges from 0.1% to 10% by weight.

The amino acids, if present, function to further preserve viral infectivity upon cooling and thawing of the aqueous suspension. In addition, amino acids function to further preserve viral infectivity during sublimation of the cooled aqueous suspension and while in the lyophilized state. A preferred amino acid is arginine, but other amino acids such as lysine, ornithine, serine, glycine, glutamine, asparagine, glutamic acid or aspartic acid can also be used. A particularly preferred arginine concentration is 0.1% by weight. Preferably, the amino acid concentration ranges from 0.1% to 10% by weight. The buffering component acts to buffer the solution by maintaining a relatively constant pH. A variety of buffers may be used, depending on the pH range desired, preferably between 7.0 and 7.8. Suitable buffers include phosphate buffer and citrate buffer. A particularly preferred pH of the FIV vector particle formulation is 7.4, and a preferred buffer is tromethamine.

In addition, it is preferable that the aqueous solution contain a neutral salt which is used to adjust the final formulated FIV vector particle to an appropriate iso-osmotic salt concentration. Suitable neutral salts include sodium chloride, potassium chloride or magnesium chloride. A preferred salt is sodium chloride.

Aqueous solutions containing the desired concentration of the components described above may be prepared as concentrated stock solutions.

One method of preserving FIV vector particles in a lyophilized state for subsequent reconstitution comprises the steps of (a) combining an infectious FIV vector particle with an aqueous solution to form an aqueous suspension, the aqueous suspension including 4% by weight of lactose, 0.1% by weight of human serum albumin, 0.03% or less by weight of NaCl, 0.1% by weight of arginine, and an amount of tromethamine buffer effective to provide a pH of the aqueous suspension of approximately 7.4, thereby stabilizing the infectious FIV vector particle; (b) cooling the suspension to a temperature of from −40° C. to −45° C. to form a frozen suspension; and (c) removing water from the frozen suspension by sublimation to form a lyophilized composition having less than 2% water by weight of the lyophilized composition, the composition being capable of infecting mammalian cells upon reconstitution. It is preferred that the FIV vector particle be replication defective and suitable for administration into humans upon reconstitution.

It will be evident to those skilled in the art given the disclosure provided herein that it may be preferable to utilize certain saccharides within the aqueous solution when the lyophilized lentivirus is intended for storage at room temperature. More specifically, it is preferable to utilize disaccharides, such as lactose or trehalose, particularly for storage at room temperature.

The lyophilized or dehydrated lentiviruses of the subject invention may be reconstituted using a variety of substances, but are preferably reconstituted using water. In certain instances, dilute salt solutions which bring the final formulation to isotonicity may also be used. In addition, it may be advantageous to use aqueous solutions containing components known to enhance the activity of the reconstituted lentivirus. Such components include cytokines, such as IL-2, polycations, such as protamine sulfate, or other components which enhance the transduction efficiency of the reconstituted lentivirus. Lyophilized or dehydrated FIV vector particle may be reconstituted with any convenient volume of water or the reconstituting agents noted above that allow substantial, and preferably total solubilization of the lyophilized or dehydrated sample.

Administration

As noted above, high titer recombinant FIV-based particles of the present invention may be administered to a wide variety of locations including, for example, into sites such as the cerebral spinal fluid, bone marrow, joints, arterial endothelial cells, rectum, buccal/sublingual, vagina, the lymph system, to an organ selected from the group consisting of lung, liver, spleen, skin, blood and brain, or to a site selected from the group consisting of tumors and interstitial spaces. Within other embodiments, the FIV vector particle may be administered intraocularly, intranasally, sublinually, orally, topically, intravesically, intrathecally, topically, intravenously, intraperitoneally, intracranially, intramuscularly, or subcutaneously. Other representative routes of administration include gastroscopy, ECRP and colonoscopy, which do not require full operating procedures and hospitalization, but may require the presence of medical personnel.

Considerations for administering the compositions of the present invention include the following:

Oral administration is easy and convenient, economical (no sterility required), safe (over dosage can be treated in most cases), and permits controlled release of the active ingredient of the composition (the lentiviral vector particle). Conversely, there may be local irritation such as nausea, vomiting or diarrhea, erratic absorption for poorly soluble drugs, and the FIV vector particle will be subject to “first pass effect” by hepatic metabolism and gastric acid and enzymatic degradation. Further, there can be slow onset of action, efficient plasma levels may not be reached, a patient's cooperation is required, and food can affect absorption. Preferred embodiments of the present invention include the oral administration of FIV vector particles that express genes encoding erythropoietin, insulin, GM-CSF cytokines, various polypeptides or peptide hormones, their agonists or antagonists, where these hormones can be derived from tissues such as the pituitary, hypothalamus, kidney, endothelial cells, liver, pancreas, bone, hemopoetic marrow, and adrenal. Such polypeptides can be used for induction of growth, regression of tissue, suppression of immune responses, apoptosis, gene expression, blocking receptor-ligand interaction, immune responses and can be treatment for certain anemias, diabetes, infections, high blood pressure, abnormal blood chemistry or chemistries (e.g., elevated blood cholesterol, deficiency of blood clotting factors, elevated LDL with lowered HDL), levels of Alzheimer associated amaloid protein, bone erosion/calcium deposition, and controlling levels of various metabolites such as steroid hormones, purines, and pyrimidines. Preferably, the FIV vector particles are first lyophilized, then filled into capsules and administered.

Buccal/sublingual administration is a convenient method of administration that provides rapid onset of action of the active component(s) of the composition, and avoids first pass metabolism. Thus, there is no gastric acid or enzymatic degradation, and the absorption of FIV vector particles is feasible. There is high bioavailability, and virtually immediate cessation of treatment is possible. Conversely, such administration is limited to relatively low dosages (typically about 10-15 mg), and there can be no simultaneous eating, drinking or swallowing. Preferred embodiments of the present invention include the buccal/sublingual administration of FIV vector particles that contain genes encoding self and/or foreign MHC, or immune modulators, for the treatment of oral cancer; the treatment of Sjogren's syndrome via the buccal/sublingual administration of such lentiviral vector particles that contain IgA or IgE antisense genes; and, the treatment of gingivitis and periodontitis via the buccal/sublingual administration of IgG or cytokine antisense genes.

Rectal administration provides a negligible first pass metabolism effect (there is a good blood/lymph vessel supply, and absorbed materials drain directly into the inferior vena cava), and the method is suitable of children, patients with emesis, and the unconscious. The method avoids gastric acid and enzymatic degradation, and the ionization of a composition will not change because the rectal fluid has no buffer capacity (pH 6.8; charged compositions absorb best). Conversely, there may be slow, poor or erratic absorption, irritation, degradation by bacterial flora, and there is a small absorption surface (about 0.05 m²). Further, lipidophilic and water soluble compounds are preferred for absorption by the rectal mucosa, and absorption enhancers (e.g., salts, EDTA, NSAID) may be necessary. Preferred embodiments of the present invention include the rectal administration of FIV vector particles that contain genes encoding colon cancer antigens, self and/or foreign MHC, or immune modulators.

Nasal administration avoids first pass metabolism, and gastric acid and enzymatic degradation, and is convenient. In a preferred embodiment, nasal administration is useful for FIV vector particle administration wherein the FIV vector particle is capable of expressing a polypeptide with properties as described herein. Conversely, such administration can cause local irritation, and absorption can be dependent upon the state of the nasal mucosa.

Pulmonary administration also avoids first pass metabolism, and gastric acid and enzymatic degradation, and is convenient. Further, pulmonary administration permits localized actions that minimize systemic side effects and the dosage required for effectiveness, and there can be rapid onset of action and self-medication. Conversely, at times only a small portion of the administered composition reaches the brochioli/alveoli, there can be local irritation, and overdosing is possible. Further, patient cooperation and understanding is preferred, and the propellant for dosing may have toxic effects. Preferred embodiments of the present invention include the pulmonary administration of FIV vector particles that express genes encoding IgA or IgE for the treatment of conditions such as asthma, hay fever, allergic alveolitis or fibrosing alveolitis, the CFTR gene for the treatment of cystic fibrosis, and protease and collagenous inhibitors such as a-1-antitrypsin for the treatment of emphysema. Alternatively, many of the same types of polypeptides or peptides listed above for oral administration may be used.

Ophthalmic administration provides local action, and permit prolonged action where the administration is via inserts. Further, avoids first pass metabolism, and gastric acid and enzymatic degradation, and permits self-administration via the use of eye-drops or contact lens-like inserts. Conversely, the administration is not always efficient, because the administration induces tearing. Preferred embodiments of the present invention include the ophthalmic administration of FIV vector particles that express genes encoding IgA or IgE for the treatment of hay fever conjunctivitis or vernal and atomic conjunctivitis; and ophthalmic administration of FIV vector particles that contain genes encoding melanoma specific antigens (such as high molecular weight-melanoma associated antigen), self and/or foreign MHC, or immune modulators.

Transdermal administration permits rapid cessation of treatment and prolonged action leading to good compliance. Further, local treatment is possible, and avoids first pass metabolism, and gastric acid and enzymatic degradation. Conversely, such administration may cause local irritation, is particularly susceptible to tolerance development, and is typically not preferred for highly potent compositions. Preferred embodiments of the present invention include the transdermal administration of FIV vector particles that express genes encoding IgA or IgE for the treatment of conditions such as atopic dermatitis and other skin allergies; and transdermal administration of FIV vector particles encoding genes encoding melanoma specific antigens (such as high molecular weight-melanoma associated antigen), self and/or foreign MHC, or immune modulators.

Vaginal administration provides local treatment and one preferred route for hormonal administration. Further, such administration avoids first pass metabolism, and gastric acid and enzymatic degradation, and is preferred for administration of compositions wherein the FIV vector particles express peptides. Preferred embodiments of the present invention include the vaginal administration of FIV vector particles that express genes encoding self and/or foreign MHC, or immune modulators. Other preferred embodiments include the vaginal administration of genes encoding the components of sperm such as histone, flagellin, etc., to promote the production of sperm-specific antibodies and thereby prevent pregnancy. This effect may be reversed, and/or pregnancy in some women may be enhanced, by delivering FIV vector particles vectors encoding immunoglobulin antisense genes, which genes interfere with the production of sperm-specific antibodies.

Intravesical administration permits local treatment for urogenital problems, avoiding systemic side effects and avoiding first pass metabolism, and gastric acid and enzymatic degradation. Conversely, the method requires urethral catheterization and requires a highly skilled staff. Preferred embodiments of the present invention include intravesical administration of FIV vector particle encoding antitumor genes such as a prodrug activation gene such thymidine kinase or various immunomodulatory molecules such as cytokines.

Endoscopic retrograde cystopancreatography (ERCP) (goes through the mouth; does not require piercing of the skin) takes advantage of extended gastroscopy, and permits selective access to the biliary tract and the pancreatic duct. Conversely, the method requires a highly skilled staff, and is unpleasant for the patient.

Many of the routes of administration described herein (e.g., into the CSF, into bone marrow, into joints, intravenous, intra-arterial, intracranial intramuscular, subcutaneous, into various organs, intra-tumor, into the interstitial spaces, intra-peritoneal, intralymphatic, or into a capillary bed) may be accomplished simply by direct administration using a needle, catheter or related device. In particular, within certain embodiments of the invention, one or more dosages may be administered directly in the indicated manner at dosages greater than or equal to 10³, 10⁴, 10⁵, 10⁶, 10⁷, 10⁸, 10⁹, 10¹⁰ or 10¹¹ cfu.

FIV vector particle may be delivered to the target from outside of the body (as an outpatient procedure) or as a surgical procedure, where the vector is administered as part of a procedure with other purposes, or as a procedure designed expressly to administer the vector. Other routes and methods for administration include the non-parenteral routes disclosed within U.S. application Ser. No. 08/366,788, filed Dec. 30, 1994, as well as administration via multiple sites as disclosed within U.S. application Ser. No. 08/366,784, filed Dec. 30, 1994.

The following examples are offered by way of illustration, and not by way of limitation.

### EXAMPLES

The following examples describe the construction of a three-plasmid viral vector system based on FIV. The first construct series described are the FIV vector constructs which contain FIV cis-acting sequences and unique cloning sites for the introduction of one or more genes of interest. FIV vector/reporter gene constructs are FIV vector constructs which may contain marker genes such as the β-galactosidase (β-gal) gene or human placental alkaline phosphatase (PLAP) gene, the expression of which is easily assayed. The second construct series described are the FIV packaging expression cassettes which provide, with the exception of the FIV envelope protein, the structural, enzymatic and regulatory proteins of FIV. The third component in the three-plasmid vector system is the env expression cassette which may express either the FIV envelope protein or a heterologous envelope protein such as the VSV-G envelope protein. Included in the following examples are also methods for vector particle production, transduction of target cells and assays for transgene expression.

All constructs were generated using standard molecular biology techniques as described in Sambrook et al., Molecular Cloning: A Laboratory Manual, 2nd ed., Cold Spring Harbor Laboratory Press, 1989). Plasmid DNA was transformed and grown in E. coli HB101 cells and isolated by passage over Qiagen mini- or giga-columns according to manufacturer's instructions. Mutations were introduced using the polymerase chain reaction (PCR), dut⁻, ung⁻ mutagenesis (Muta-gene Kit, BioRad Laboratories, Hercules, Calif.; Kunkle, PNAS 82: 488, 1985) or the Quick-Change In Vitro Mutagenesis Kit (Stratagene, San Diego, Calif.) with oligonucleotides synthesized by Operon Technologies Inc. (Alameda, Calif.). All plasmids were screened by restriction enzyme digestion and their nucleotide sequence confirmed by sequence analysis (SeqWrite, LLC, Houston, Tex.).

### Example 1

### Construction of FIV Vectors

FIV vector, or pTFIV, constructs were generated in a series of steps from FIV-34TF10 (FIV proviral DNA) which will henceforth be referred to as pF34. pF34 was obtained from NIH AIDS Research and Reference Reagent Program (FIV-34TF10, Cat. No. 1236; Phillips et al., J. Virol. 66: 5464, 1992, Talbott et al., PNAS 86: 5743, 1989) and contains a 9.5 kb (9472 bp) insert from FIV-Petaluma plus 0.2 kb each of 5′ and 3′ flanking cellular DNA (the sequences of which are recorded as SEQ ID No. 1 and 2, respectively) cloned into pUC119. pTFIV constructs consist of the 5′ and 3′ FIV LTRs from pF34 and some portion of the non-coding region immediately following the 5′ FIV LTR. This portion of the non-coding region includes the first splice donor site and likely includes some part of the putative FIV packaging signal. In addition, pTFIV constructs may contain some portion of the FIV Gag coding region as well as the FIV RRE. The term ‘pTFIV construct’ encompasses two series of constructs, the pTFIVS series and pTFIVL series, which differ by containing either a short (S) or long (L) segment corresponding to the Gag coding region.

A. Construction of the pTFIVS Vector

In general, to construct the pTFIVS vector, DNA corresponding to the 5′ FIV LTR plus a portion of the Gag ORF was amplified from pF34 by PCR and cloned into an intermediate plasmid. Likewise, DNA corresponding to the 3′ FIV LTR plus the FIV RRE was amplified from pF34 by PCR and also cloned into an intermediate plasmid. The 5′ FIV LTR fragment was then released from the intermediate construct and ligated into the 3′ FIV LTR-containing intermediate plasmid to create the pTFIVS vector. More specifically, to generate the 5′ region of pTFIVS, FIV primers FIV13 (SEQ ID No. 3) and FIV14 (SEQ ID No. 4) were used to PCR-amplify a fragment corresponding to the 5′ LTR and a 0.35 kb portion of the Gag coding region. FIV13 (TTC ATA CCG CGG TGG GAT GAG TAC TGG AAC C) corresponds to the 5′ FIV LTR from nt 1 through nt 31 and contains a Sac II site (underlined) near its 5′ end. FIV 14 (CAA ATA GCG GCC GCA GCA GCA GTA GAC ACC) is complementary to a region of the Gag ORF which includes the TthIII 1 site at nt 920 and contains an additional Not I site (underlined) near its 5′ end. To generate the 3′ region of pTFIVS, primers FIV16 (SEQ ID No. 6) and FIV18 (SEQ ID No. 7) were used to amplify a fragment corresponding to the 3′ FIV LTR and adjacent RRE. FIV16 (GTT AAC GGG CCC AAG AAA TAC AAC CAC AAA TGG) corresponds to FIV nt 8761 through 8781 and contains an Apa I site (underlined) near its 5′ terminus. FIV 18 (ATC GAT GGT ACC TGC GAA GTT CTC GGC CC) corresponds to the FIV 3′ LTR from nt 9443 to nt 9472 and includes a Kpn I site near its 5′ terminus. PCR samples contained 100 pmol of each primer, 200 M each dNTP, 2 U Pfu DNA polymerase (Stratagene, San Diego, Calif.), 10 l 10× Pfu buffer and 50 ng pF34 DNA as template. PCR samples were denatured at 95° C. for 2 min then subjected to 25 cycles of denaturation, annealing and extension conditions consisting of 95 C for 2 min, 55 C for 0.5 min and 72° C. for 1 min or longer (i.e. 30 sec for each 400 bases to be amplified), respectively. After 25 cycles, reactions were held at 72° C. for 10 min to favor complete extension and then kept at 4° C. for 5 min to overnight. PCR products were gel-purified and ligated directly into pPCR-Script SK (+) (Stratagene, San Diego, Calif.) to generate pCR13/14 and pCR16/17. pCR16/17 was digested with Kpn I and Apa I and the liberated fragment ligated into similarly digested pBlueScript KS II (+) to create pB3′ FIV. pCR13/14 was digested with Sac II and Not I and the resulting fragment ligated into similarly digested pB3′ FIV to create pTFIVS.

B. Construction of the pTFIVL Vector

The pTFIVL vector was constructed in a manner similar to that of the pTFIVS vector; i.e. the 5′ LTR and 3′ LTR portions were individually amplified by PCR, cloned into intermediate plasmids, then combined to form the complete pTFIVL vector. The 3′ region of pTFIVL is identical to that of pTFIVS and was generated as described in example 1A. The 5′ region of pTFIVL was generated using FIV primers FIV13 (example 1A) and FIV15 (SEQ ID No. 5) to amplify a fragment corresponding to the 5′ LTR plus a 0.55 kb portion of the Gag coding region. FIV15 (CAA ATA GCG GCC GCG TTG AAC TTC CTC ACC TCC) is complementary to a region of the Gag ORF from nt 1107 to nt 1140 and contains an additional Not I site (underlined) near its 5′ terminus. PCR products were gel-purified and ligated directly into pPCR-Script SK (+) to generate pCR13/15 and pCR16/17 (example 1A). pCR13/15 was digested with Sac II and Not I and the resulting fragment ligated into similarly digested pB3′ FIV (example 1A) to create pTFIVL.

### Example 2

### Construction of Hybrid FIV LTR Vectors

Hybrid FIV LTR vectors are similar to the FIV vectors described in example 1, however the hybrid vectors contain heterologous enhancer and/or promoter elements in place of all or part of the U3 region of the 5′ FIV proviral DNA LTR. The pTC/FIV constructs, described below, are similar to the pTFIV series but contain CMV promoter/enhancer elements in place of the FIV U3 region. pTC/FIVS is analogous to pTFIVS with respect to containing a short portion of the Gag coding region while pTC/FIVL is analogous to pTFIVL in containing a long portion of the Gag coding region downstream of the 5′ FIV LTR.

A. Construction of the pTC/FIVS Hybrid FIV LTR Vector

pTC/FIVS, in which the FIV U3 region has been replaced by the CMV promoter/enhancer, was generated using the ‘sewing PCR’ method of Deminie and Emerman (J. Virol. 67: 6499, 1993). Briefly, this method consists of two rounds of PCR, the first round generating two or three PCR fragments with overlapping regions which are subsequently annealed to one another to serve as template DNA for the second round PCR. For first round PCR, primers FIV19 (SEQ ID No. 8) and FIV20 (SEQ ID No. 9) were used to amplify the region corresponding to the CMV promoter/enhancer from pCMV (Clontech Laboratories Inc., Palo Alto, Calif.). In a separate reaction, primers FIV21 (SEQ ID No. 10) and FIV14 (see example 1A) were used to generate the FIV U3 and R region from pF34 template DNA (see PCR conditions in example 1A). FIV19 (CCG CGG GAG CTT GCA TGC CTG CAG) corresponds to the CMV enhancer region of pCMV from nt 1 to nt 24 and but contains a Sac II site (underlined) in place of the EcoR I site at nt 1. The 5′ end of FIV20 (TTT CAC AAA GCA CTG GTT ATA TAG ACC TCC CAC CG) is complementary to a region of the CMV promoter up to and including the TATA box (underlined) and the 3′ end is complementary to the FIV R region (italicized). The 5′ end of FIV21 (CGG TGG GAG GTC TAT ATA ACC AGT GCT TTG TGA AA) corresponds to the CMV promoter and TATA box (underlined) and the 3′ end corresponds the FIV R region (italicized), thus FIV 21 is complementary to FIV20. FIV14 has been described previously (see example 1A). For second round PCR, the FIV 19/20 and FIV 21/14 PCR fragments were gel-purified and 5 l of each used as template DNA for the amplification of a CMV/FIV hybrid LTR using FIV19 and FIV14 as primers. The second round PCR product was ligated directly into pPCR-Script SK(+) (Stratagene, San Digo, Calif.) to create pCR19/14. pCR19/14 was then digested with Sac II and Not I and the resulting 1.3 kb fragment ligated into similarly digested pB3′ FIV to create pTC/FIVS.

B. Construction of the pTC/FIVL Hybrid FIV LTR Vector

The pTC/FIVL hybrid vector is identical to pTC/FIVS except that pTC/FIVL contains a long portion of the Gag coding region downstream of the 5′ FIV LTR. pTC/FIVL was constructed in parallel with pTC/FIVS using the methods described in example 2A. Briefly, to create pTC/FIVL, primer FIV 15 (example 1 B) was used in place of primer FIV14 to generate the FIV U3 and R region from pF34 template DNA during first round PCR. For second round PCR, the resulting FIV 21/15 fragment was gel-purified and used together with the FIV 19/20 fragment (example 2B) and primers FIV 19 and 15 to amplify the CMV/FIV hybrid LTR. The resulting second round PCR product was ligated directly into pPCR-Script SK(+) to create pCR19/15 was then digested with Sac II and Not I and the resulting 1.5 kb fragment ligated into similarly digested pB3′ FIV to create pTC/FIVL.

### Example 3

### Insertion of Promoter/Reporter Gene Cassettes into FIV Vectors

Promoter/reporter gene cassettes consist of a heterologous promoter (e.g. the CMV or SV40 promoter) followed by a reporter gene such as the -galactosidase (-gal) gene or human placental alkaline phospatase gene (PLAP). Such cassettes were generated and inserted into one or more FIV vectors or hybrid FIV LTR vectors to create FIV/reporter gene vectors. FIV/reporter gene vectors may contain the FIV RRE and, in addition, may contain heterologous export elements (HEEs) such as the MPMV CTE or HBV PRE (see detailed description). FIV/reporter gene vectors (e.g. pTFSCCTE) are named according to the vector backbone (e.g. pTFIVS, in this case shortened as pTFS), the heterologous promoter (e.g. CMV, denoted by C), the reporter gene(s) within the cassette (e.g. -gal or ) and the heterologous export element (e.g. CTE), if present.

A. Generation of the pCMVgal Expression Cassette

To generate pCMVgal, a 0.75 kb fragment containing the hCMV (henceforth referred to as CMV) early gene promoter was first liberated from pCMV-G (Yee et. al., PNAS 91:9564, 1994) by digestion with Xba I and Sal I. Next, a 3.1 kb Sal I/Sma I fragment containing the -gal gene was released from pUCgal . pUCgal contains the Xba I/SacI and SacI/SmaI-gal gene fragments from pSP6-GAL (Xu et al., Virology 171:331, 1989) cloned into Xba I/Sma I digested pUC 19 (Clontech Laboratories, Inc. Palo Alto, Calif.). Finally, the 0.75 kb CMV promoter fragment from pCMV-G and the 3.1 kb -gal gene fragment from pUCgal were gel-purified, ligated together and inserted into Xba I/Sma I digested pBluescript SK (−) to create pCMVgal.

B. Generation of the pCMVgalCTE Expression Cassette

The construction of pCMVgalCTE was accomplished after amplification of the CTE by PCR from MPMV using the oligos CTEH5 (GTC AAG CTT AGA CTG GAC AGC CAA TG) and CTEH3 (CTA AAG CTT CCA AGA CAT CAT CCG GG) which harbor Hind III sites near their 5′ ends (underlined). The PCR product was digested with Hind III and inserted into the Hind III site of pBluescript SK (−) to create pSK-CTE. pSK-CTE was then digested with Sma I and Xho I and the resulting 0.2 kb fragment ligated into similarly digested pCMVgal (example 3A) to create pCMVgalCTE.

C. Generation of the pCMVgalPRE Expression Cassette

To generate pCMVgalPRE, a 0.65 kb fragment was released from pCCAT-1 (Yee, J-K. Science 246: 658, 1989) by digestion with Stu I and Hind III. The 0.65 kb fragment was treated with the Klenow fragment of DNA Polymerase I and ligated into the EcoRV site of pBluescript SK (−) to create pSK-PRE. pSK-PRE was then digested with Sma I and Xho I and the resulting 0.66 kb fragment ligated into similarly digested pCMVgal (example 3A) to create pCMVgalPRE.

D. Generation of the pCMVgalRRE Expression Cassette

pCMVgalRRE was generated in a manner similar to that described for pCMVgalCTE (example 3B). The HIV-1 RRE was amplified by PCR from the molecular clone pNL4-3 (Adachi et al., J. Virol. 59: 284, 1986) using the oligos RRE1 (GCA AGC TTC TGC AGA GCA GTG GGA ATA GG) and RRE2 (GCA AGC TTA CCC CAA ATC CCC AGG AGC TG) which harbor Hind III sites near their 5′ ends (underlined). The amplified product was digested with Hind III and inserted into the Hind III site of pBluescript SK (−) to create pSK-RRE. pSK-RRE was then digested with Sma I and Xho I and the resulting fragment ligated into similarly digested pCMVgal to create pCMVgalRRE

E. Construction of the pTFSCFIV Vector

pCMVgalCTE (example 3B), containing the CMV promoter/enhancer, -gal gene and CTE element was the source of reporter gene expression cassette for the construction of the pTFSC vector. To create pTFSC pCMVgalCTE was digested with Not I and Sma I and the resulting 3.8 kb fragment (containing the CMV promoter and -gal gene) gel-purified and ligated into similarly digested pTFIVS.

F. Construction of the pTFLCFIV Vector

To create pTFLC, pCMVgalCTE (example 3B) was digested with Not I and Sma I and the resulting 3.8 kb fragment gel-purified and ligated into similarly digested pTFIVL.

G. Construction of the pTFSCCTEFIV Vector

To create pTFSCCTE, pCMVgalCTE (example 3B), was digested with Not I and Xho I and the resulting 4.0 kb fragment (containing the CMV promoter, -gal gene and CTE element) gel-purified and ligated into Not I/Sal I digested pTFIVS.

H. Construction of the pTFLCCTEFIV Vector

To create pTFLCCTE, pCMVgalCTE (example 3B), was digested with Not I and Xho I and the resulting 4.0 kb fragment (containing the CMV promoter, -gal gene and CTE element) gel-purified and ligated into Not I/Sal I digested pTFIVL.

I. Construction of the pTFSCPREFIV Vector

To createpTFSCPRE, pCMVgalPRE (example 3C) was the source of reporter gene expression cassette. pCMVgalPRE was digested with Not I and Xho I and the resulting 4.5 kb fragment (containing the CMV promoter, -gal gene and PRE element) gel-purified and ligated into Not I/Sal I digested pTFIVS.

J. Construction of the pTFLCPREFIV Vector

To create pTFLCPRE, pCMVgalPRE (example 3C) was digested with Not I and Xho I and the resulting 4.5 kb fragment (containing the CMV promoter, -gal gene and PRE element) gel-purified and ligated into Not I/Sal I digested pTFIVL.

K. Construction of the pTFSCRREFIV Vector

To create pTFSCRRE, pCMVgalRRE (example 3D) was the source of reporter gene expression cassette. pCMVgalRRE was digested with Not I and Xho I and the resulting 4.3 kb fragment (containing the CMV promoter, -gal gene and RRE element) gel-purified and ligated into Not I/Sal I digested pTFIVS.

L. Construction of the pTFLCRREFIV Vector

To create pTFLCRRE, pCMVgalRRE (example 3D) was digested with Not I and Xho I and the resulting 4.3 kb fragment (containing the CMV promoter, -gal gene and RRE element) gel-purified and ligated into Not I/Sal I digested pTFIVL.

M. Construction of the pTC/FSChybrid FIV LTR Vector

To create pTC/FSC, pCMVgalCTE (example 3B) was digested with Not I and Sma I and the resulting 3.8 kb fragment (containing the CMV promoter and -gal gene) gel-purified and ligated into similarly digested pTC/FIVS.

N. Construction of the pTC/FLChybrid FIV LTR Vector

To create pTC/FLC, pCMVgalCTE (example 3B) was digested with Not I and Sma I and the resulting 3.8 kb fragment (containing the CMV promoter and -gal gene) gel-purified and ligated into similarly digested pTC/FIVL.

### Example 4

### Insertion of Reporter Gene Cassettes into FIV Vectors

To generate FIV vectors containing heterologous genes but lacking heterologous promoters to drive the transcription of such genes, FIV vectors were generated in which transcription of the heterologous gene (e.g. reporter gene) is driven by the FIV 5′ LTR.

A. Construction of the pTFS FIV Vector

To create pTFS pTFIVS (example 1A) was digested with Xba I and Sma I and ligated together with the 3.1 kb Xba I/Sma I fragment containing the -gal gene from pCMVgal (example 3A).

B. Construction of the pTFL FIV Vector

To create pTFL pTFIVL (example 1B) was digested with Xba I and Sma I and ligated together with the 3.1 kb Xba I/Sma I fragment containing the -gal gene from pCMVgal (example 3A).

### Example 5

### Construction of FIV Packaging Expression Cassettes

The FIV packaging expression cassettes (pCMVFIV constructs) contain the FIV gag, pol, vif, rev and ORF 2, flanked by the CMV promoter at the 5′ end and SV40 polyadenylation signal at the 3′ end. The pCMVFIV packaging constructs were generated in a series of steps beginning with the deletion of a 1.6 kb region corresponding to the FIV env gene in pF34. Briefly, pF34 was digested with Kpn I and Spe I and the 1.9 kb env fragment inserted into similarly digested pBluescript II KS(+) to generate pBF34env. pBF34env was digested with Avr II and Spe I, releasing a 1.6 kb product, and religated to generate pBF34env. pBF34env was then digested with Kpn I and Xba I and the resulting 0.3 kb product gel purified and ligated into Kpn I/Spe I digested pF34 to create pF34env (FIVenv provirus). pF34env was then used as the source of FIV sequences for constructing the following pCMVFIV packaging cassettes.

The pCMVFIV packaging constructs described below, differ by containing various lengths of sequence corresponding to the FIV 5′ noncoding region downstream of the 5′ FIV LTR. pCMVFIVXho was constructed using a convenient Xho I site located at nt 500 and therefore contains 0.1 kb of noncoding sequence upstream of the FIV (SD₆₀₄) 5′ splice donor site (i.e. lacks 0. 14 kb of the 0.24 kb noncoding sequence between the 3′ border of the 5′ LTR and the 5′ splice donor site). pCMVFIVSal was created after the introduction of a Sal I site at nt 578 and therefore contains only 0.02 kb of noncoding sequence (i.e. lacks 0.22 kb of the noncoding sequence). The 17 mutation in pCMVFIV17 and pCMVFIVSal17 refers to a deletion of 17 bp in the sequence corresponding to the region between the FIV 5′ splice donor and the ATG codon of gag.

A. Construction of Packaging Expression Cassette, pCMVFIVXho

To generate pCMVFIVXho from pF34env, a Not I restriction enzyme recognition site was first introduced into pF34env at nt 9168 by oligonucleotide directed in vitro mutagenesis using two rounds of PCR. The first round PCR contained 200 M each dNTP, 2 U Pfu DNA polymerase, 10 l 10× Pfu buffer, 50 ng template DNA (pF34 plasmid DNA and 100 pmol each of primers FIV5 (SEQ ID NO. 11; AAA TGG TAG GCA ATG TGG C) and FIV6 (SEQ ID NO. 12; CCT TTT ATC ATT TGT TCG TAA GCG GCC GCT AGT CCA TAA GCA TTC TTT C) or, in a separate reaction, 100 pmol each of primers FIV7 (SEQ ID No. 13; GAA AGA ATG CTT ATG GAC TAG CGG CCG CTT ACG AAC AAA TGA TAA AAG G) and FIV8 (SEQ ID No. 14; CAC TTT ATG CTT CCG GCT C). PCR samples were denatured at 95 C for 2 min then subjected to 25 cycles of denaturation, annealing and extension conditions of 95 C for 2 min, 55 C for 30 sec and 72 C for 1 min or longer (i.e. 30s for each 400 bases to be amplified), respectively. After 25 cycles, reactions were held at 72° C. for 10 min to favor complete extension and then kept at 4° C. for 5 min to overnight. The second round PCR was identical to the first but with 5 l gel each gel-purified PCR product serving as template DNA (either the 0.38 kb FIV 5/6 fragment or the 0.6 kb FIV 7/8 fragment) and oligos FIV5 and FIV8 serving as primers. The 0.95 kb second round PCR product was purified, cleaved with Nde I and Sal I, and the resulting 0.74 kb product ligated into similarly digested pF34env to generate pF34Nenv. pF34Nenv was then digested either with TthIII 1 and Not I to obtain a 6.7 kb fragment or Xho I and TthIII 1 to generate a 0.4 kb product. The purified 6.7 kb and 0.4 kb products were ligated together with a purified 3.6 kb Not I/Xho I fragment from pCMV to create pCMVFIVXho.

B. Construction of Packaging Expression Cassette, pCMVFIVSal

To generate pCMVFIVSal, a Sal I restriction enzyme recognition site was first introduced into pF34Nenv by in vitro mutagenesis as described above. The first round PCR contained either oligos FIV1 (SEQ ID No. 15; TGA GGA AGT GAA GCT AGA GC) and FIV2 (SEQ ID No. 16; GTT GAC TGT CCC TCG GCG AGT CGA CTG GCT TGA AGG TCC GCG) or oligos FIV 3 (SEQ ID No. 17; CGC GGA CCT TCA AGC CAG TCG ACT CGC CGA GGG ACA GTC AAC) and FIV4 (SEQ ID No. 18; TTG AAC TTC CTC ACC TCC TAG) and generated either a 0.2 kb or 0.54 kb PCR product, respectively. The second round PCR, containing the purified first round products and oligos FIV 1 and FIV4, gave rise to a 0.75 kb product. The purified second round product was digested with TthIII 1 and Sac I and the resulting 0.4 kb product ligated into similarly digested pF34Nenv to create pF34NSenv. pF34NSenv was then cleaved with Sal I and Not I and ligated into Xho I/NotI digested pCMV to create pCMVFIVSal.

C. Construction of Packaging Expression Cassette, pCMVFIV17S

To generate pCMVFIV17S, in vitro mutagenesis was carried out either using oligos FIV1 (example 5B) and FIV9 (SEQ ID No. 19; CCC CTG TCC ATT CCC CAT CCT ACC TTG TYG ACT GTC CCT CGG CGA A where Y is C or T) or using oligos FIV10 (SEQ ID No. 20; GGA CAG TCR ACA AGG TAG GAT GGG GAA TGG ACA GGG G where R is A or G) and FIV4 (example 5B) in the first round PCR. The second round PCR contained the 0.23 kb and 0.53 kb products resulting from first round PCR and oligos FIV1 and FIV4. The 0.73 kb second round PCR product was then digested with Sac I and TthIII 1 and ligated into similarly digested pF34Nenv to generate pF34N17Senv. As above, this latter product was cleaved with Sal I and Not I and ligated into Xho I/Not I digested pCMV to generate pCMVFIV17S.

D. Construction of Packaging Expression Cassette, pCMVFIVSal17

A construct similar to pCMVFIV17S, described above, pCMVFIVSal17, was generated by virtue of oligo FIV9 (example 5C) being a degenerate oligo (which may or may not cause the introduction of a Sal I site during in vitro mutagenesis). By using the degenerate oligo FIV9 as a primer (along with FIV1; example 5B) and pF34NSenv as the DNA template for first round PCR (as described in example 5C), the 17 mutation could be made without the introduction of an adjacent Sal I site. The 0.73 kb second round PCR product was digested with Sac I and TthIII 1, as above, and the resulting fragment ligated into pF34Nenv to generate pF34NS 17env. This latter product was cleaved with Sal I and Not I, as above, and ligated into Xho I/Not I digested pCMV to generate pCMVFIVSal17.

### Example 6

### Production of Pseudotyped FIV Particles

FIV particles lacking the FIV envelope protein but containing the VSV-G envelope protein (i.e. pseudotyped with VSV-G Env) were produced by cotransfection of the FIV envelope deletion construct, pF34env (example 5), and a VSV-G envelope-expressing plasmid, pCMV-G (Yee et al., PNAS 91: 9564, 1994) into Crandell feline kidney (CrFK) cells. Calcium phosphate-DNA complexes were prepared using the Profectin kit (Promega Corp. Madison, Wis.) according to the manufacturer's instructions using a 1:1 ratio of pF34env and pCMV-G plasmid DNA. Following transfection, the cells were placed in a 5% CO₂ incubator for 6 hr. to overnight afterwhich the medium was replaced and the cells returned to 10% CO₂ for an additional 36 to 66 hr. (i.e. 48 to 72 hr. following transfection). The supernatant was then harvested, filtered through a 0.45 M Nalgene filter and either used immediately for infection or frozen at −70 C until further use.

### Example 7

### Infection of Cultured Cells by Pseudotyped FIV Particles

Serial dilutions of supernatant containing pseudotyped FIV particles (example 6) were incubated with CrFK, HT1080, or 293 cells in culture medium containing 8 g/ml polybrene. After 12 to 24 hr incubation, the culture medium was removed, the cells washed three times with PBS, and then maintained in DMEM supplemented with 10% FBS for an additional 24 to 60 hr (i.e. 48 to 72 hr after initial infection) at 10% CO₂. The supernatant was then removed and assayed for the presence of the FIV major core protein (Gag) using the PetCheck FIV Antigen Test Kit (IDEXX, Portland, Me.) according to manufacturer's instructions. The presence of FIV p24 (referred to by its original designation of p26 in the IDEXX kit, however, more recently designated as p24; Talbott et al., PNAS 86: 5743; Tilton et al., J. Clin. Microbiol. 28: 898), indicated that pseudotyped FIV particles can be produced by cotransfection in CrFK cells and that these particles are capable of infecting naive CrFK cells. Preliminary results are summarized in Table 1.

TABLE 1
Transduction of pF34env into human
(HT1080) and cat (CrFK) kidney cells

| | HT1080 | CRFK |
|---|---|---|
| pF34 (FIV env^{a}) | −^{b} | + |
| pF34env | − | − |
| pF34env (VSV-G^{a}) | + | +++ |

^{a}refers to the virus particle envelope protein
^{b}transduction assessed from FIV p24 levels

### Example 8

### Production of FIV Vector Particles

FIV vector particles were produced by transient triple transfection of an FIV/reporter gene vector, an FIV packaging expression construct and a VSV-G envelope-expressing plasmid into CrFK, 293 or 293T human kidney cells. DNA complexes were prepared using calcium phosphate (e.g. Profectin kit; Promega Corp. Madison, Wis.) or cationic lipid reagents (e.g. Lipofectamine Plus Reagent, Gibco BRL/Life Technologies, Rockville, Md.; Superfect Transfection Reagent, Qiagen Inc. Valencia, Calif.) and transfected into cells according to the manufacturer's instructions. Transfected cells were incubated for 24 to 72 hr. following transfection afterwhich the supernatant was harvested and filtered through a 0.45 M Nalgene filter. The vector particle-containing supernatant was either used immediately for infection or concentrated by centrifugation. Vector particles were concentrated by layering the pooled filtered supernatant over a cushion of 20% sucrose and centrifuging in a Beckman SW28 rotor at 50,000× g for 90 min at 4° C. The pellet was resuspended in PBS at 4° C. and again centrifuged at 50,000× g in a Beckman SW55 rotor for 90 min at 4° C. The pellet was resuspended in PBS and used immediately for infection or stored at −70° C. until further use.

### Example 9

### Infection of Cultured Cells by FIV Vector Particles

Serial dilutions of pseudotyped FIV vector particles (before or after concentration from transfected cell supernatants) were added to CrFK, HT1080, 293 or 293T cells in culture medium containing 8 g/ml polybrene. The cultures were incubated for 48 to 72 hr. following initial infection and then assayed for expression of the transduced gene. -galactosidase expression was assayed after removing the medium and fixing the cells in cold 2% formaldehyde/0.2% glutaraldehyde in PBS for 5 min. The cells were washed twice with PBS and stained with fresh X-gal staining solution consisting of 1 mg/ml X-gal, 5 mM potassium ferricyanide, 5 mM potassium ferrocyanide and 2 mM MgCl₂ in PBS for 50 min at 37° C. The cells were again washed with PBS and the titer determined from the number of blue foci per well.

### Example 10

### Infection of Non-Proliferating Cells by FIV Vector Particles

To test the ability of FIV vectors to transduce cells blocked at the G2 phase of the cell cycle, HeLa cells were growth arrested by exposure to gamma irradiation (Kastan et al., Cell 71: 587, 1992). The arrested state of the cells was verified by propidium iodide staining of the DNA and flow cytometry prior to infection. Pseudotyped FIV vectors capable of expressing -galactosidase were used to infect the growth-arrested cells and the transduction efficiency scored by X-gal staining (example 9) of the cultures 48 hr after infection.

To determine whether pseudotyped FIV vectors are capable of transducing non-proliferating primary cells, -galactosidase expressing FIV vectors were used to transduce human monocyte-derived macrophages. Monocytes were harvested from the blood of healthy donors and purified by centrifugation over Ficoll/Hypaque (Kombluth et. al., J. Exp. Med. 169: 1137, 1989). Monocytes were further purified by adherence to plastic and maintained in RPMI containing 10% human serum for two weeks. VSV-G pseudotyped FIV vectors capable of expressing -galactosidase were then used to infect the terminally differentiated macrophages and the transduction efficiency measured after X-gal staining (example 9).

### Example 11

### Construction of MLV-FIV Hybrid Vectors for Efficient Delivery of FIV Vector Genomes into FIV Packaging Cell Lines

In this particular example the FIV vector was constructed based on pVETS, the MLV backbone was pBA-9b and the gene of interest was EGFP. The strategy consisted of the insertion of FIV vector into a self-inactivating (sin) MLV vector that allowed for the expression of the FIV vector but prevented the expression of the MLV vector genome in the FIV packaging cell line. Additionally the FIV vector genome was inserted in the opposite orientation with respect to the direction of transcription of the MLV vector genome in order to circumvent the premature termination of MLV transcription induced by the polyadenylation signal present in the 3′ LTR of FIV.

A. Construction of pVETS-GFP

pVETS-CGFP was generated by ligating a 1874 bp NotI to HindIII fragment from pVETL-CGFP containing the CMV-GFP cassette into pVETS linearized by double digestion with NotI and HindIII.

B. Construction of a sinMLV Vector Backbone

A 677 bp NotI to EcoRI fragment from pBA-9b encompassing the 3′ LTR of MLV was cloned into pSK(−) (Stratagene, La Jolla, Calif.) digested similarly to generate pSKMLTR. To delete the MLV LTR enhancer region, pSKMLTR was digested with NheI and XbaI and self-religated to generate pSKMLTRΔN/X. The MLV LTR TATA box was mutagenized by PCR using the oligonucleotides MTmutAvr5 5′ CTTCTGCTCCCCGAGCTCCCTAGG-AGAGCCCACAACCCCTCA3′ (SEQ ID NO: 21) and MTmutAvr3 5′ TGAGGGGTTGTGGGCTCTCC-TAGGGAGCTCGGGGAGCAGAAG3′ (SEQ ID NO:22) which introduced a AvrII site in place of the TATA box. The resulting plasmid was named pSKMLTRsin.

For splice donor (SD) mutagenesis, pBA-9b was digested with Spel and EcoRI and ligated to the oligonucleotide linker EcoBSpe 5′ AATTCTAAGTATACGGCA3′ (SEQ ID NO:23) and SpeBEco 5′ CTAGTGCCGTATACTTAG3′ (SEQ ID NO:24) both of which harboring a BstZ17I site, to generate pBA-9bΔSR. This construct was submitted to PCR based mutagenesis using the oligonucleotides MSDmut5 5′ GACCACCGACCCACCACCGGTATACAAGCTGGCCAGCAACTTA3′ (SEQ ID NO:25) and MSDmut3 5′ TAAGTTGCTGGCCAGCTTGTATACCGGTGGTGGGTCGGTGGTC3′ (SEQ ID NO:26) which resulted in the introduction of a BstZ17I site in place of the MLV splice donor. The resulting construct was named pBA-9bΔSRΔSD.

The self-inactivating, splice donor defective MLV vector backbone was generated by assembling in a single step i) a 3367 bp EcoRI to SpeI fragment from pBA-9bΔSRΔSD, ii) a 788 bp SpeI to NotI fragment from pBA-9b and iii) a 410 bp NotI to EcoRI fragment from pSKMLTRsin. The resulting construct was named pBA-9b(−SD)SIN.

C. Construction of the MLV-FIV Vector Hybrid.

In order to be able to use convenient restriction enzymes, the FIV vector was cloned into pSK(−). To do so, pVETS-GFP was digested with Acc65I repaired with T4 DNA polymerase and subsequently digested with PstI. The resulting 3333 bp FIV vector fragment was introduced into pSK(−) digested with PstI and EcoRV to generate the construct labeled pSK-VCGFP. Finally the FIV vector was excised from pSK-VCGFP by digestion with BamHI and SalI and inserted into pBA-9b(−SD)SIN linearized by digestion with BamHI and XhoI, to generate pMC-GFP.

D. Production of VSV-G Pseudotyped MC-GFP Particles and Titer Determination

293T cells were transfected with 10 μg MLV packaging construct pSCV10, 5 μg pCMV-G and 15 μg pMC-GFP. Supernatant was collected every 24 hours for 2 days, precipitated with 10% PEG8000, NaCl 15 mM and vectors were resuspended in 1/24 initial volume phosphate buffered saline. To determine MC-GFP vector titer, 25, 75 and 100 μl of concentrated particles were used to transduce 2.5×10⁵ naïve HT1080 cells in the presence of 8 μg/ml polybrene and FACS analysis of GFP expresssion was performed 2 days post-transduction. GFP positive cells and titers are shown in the following Table:

| Volume of inoculum | GFP positive cells | Titer |
|---|---|---|
| (μl) | (%) | (TU/ml) |
| 25 | 3.15 | 8.6 × 10⁵ |
| 75 | 23.13 | 2.1 × 10⁶ |
| 100 | 39 | 2.6 × 10⁶ |

E. Transduction of FIV Packaging Cell Lines

The FIV vector packaging cell lines 3ΔΔ5 and 3ΔΔ6 were chosen for the generation of vector producer cell lines. 2.5×10⁵ cells were transduced with either 100, 300 or 600 μl of concentrated vector which corresponds to multiplicities of infection of 0.4, 1.2 and 2.4 respectively. Forty-eight hours post-transduction cells were passaged at a dilution of ¼ and transduced again 24 hours later with 100 μl of concentrated vector.

F. Assay for VPCL Titer Potential

Three resulting cell pools (d5MC-1, d5MC-6 and d6MC-6) were analyzed for titer potential on HT1080 cells. Briefly 2.5×10⁵ cells were transduced with either 10, 100 or 1000 μl of crude supernatant acording to the usual protocol. In parallel the same transductions were performed in the presence of 50 μg/ml 3′-azido-3′-deoxythimidine (AZT) to control for pseudotransduction. The results are as follows:

| Cell Line/ | % GFP | % GFP | | |
|---|---|---|---|---|
| vol inoculum | pos. cells | pos. cells | % Transduced | Titer |
| (ml) | w/o AZT | w/AZT | cells | (TFU/ml) |
| d5MC-1/0.01 | 4.96 | 0.07 | 4.89 | 1.2e6 |
| d5MC-1/0.1 | 6.78 | 0.25 | 6.53 | 1.6e5 |
| d5MC-1/1 | 6.05 | 0.23 | 5.82 | 1.4e4 |
| d5MC-6/0.01 | 9.05 | 0.71 | 8.34 | 2e6 |
| d5MC-6/0.1 | 7.52 | 0.89 | 6.63 | 1.6e5 |
| d5MC-6/1 | 6.48 | 0.86 | 5.62 | 1.4e4 |
| d6MC-6/0.01 | 13.41 | 0.50 | 12.91 | 3.2e6 |
| d6MC-6/0.1 | 8.46 | 0.82 | 7.64 | 1.9e5 |
| d6MC-6/1 | 6.61 | 0.68 | 5.93 | 1.5e4 |

In order to eliminate the nucleotide sequence redundancy between the CMV enhancer/promoter present in FIV 5′ LTR and that upstream the GFP gene in pVETS-GFP, the former is replaced with the enhancer/promoter of RSV.

F. Synthesis of an RSV-Hybrid FIV LTR

A 230 bp fragment encompassing the RSV enhancer/promoter was amplified from pRc/RSV (Invitrogen, Carlsbad, Calif.) with the oligonucleotides RSVSacII-5 5′ AACCGCGGAAATGTAGTCTTA-TGCAATACACTTGTAGTC3′ (SEQ ID NO: 27) harboring a SacII site at its 5′ end (bold) and RSVR-3 5′ CCTCAACA-AAGAGACTCCGTTTATTGTATCGAGCTAGGC3′ (SEQ ID NO:28) which 5′ end is complementary to the 5′ end of the R region of FIV LTR (underlined).

In parallel, a 736 bp fragment encompassing the 5′ portion of the FIV vector from the R region of the LTR down to the beginning of the multiple cloning site was amplified from pVETS with the oligonucleotides FIVR-5 5′ GGAGTCTCTTTGTTGAGGACTTTTGAGTTCTCCC3′ (SEQ ID NO:29) and VET-N3 5′ TAGAGCGGCCGCAGCAGCAGTAGACACCGTC3′ (SEQ ID NO:30) which contains a Not I site at its 5′ end (bold).

The 2 PCR fragments were linked by fusion-extension and the resulting fragment was amplified using the oligonucleotides RSVSacII-5 and VET-N3 and labeled RSV-R.

G. Construction of a FIV GFP Vector with an RSV/FIV 5′ LTR

pSK-RVCGFP is genarated by ligating in a 3-way reaction the fragment RSV-R digested with Not I, the 2105 bp NotI to SalI fragment from pSK-VCGFP and the 2931 bp SalI to SmaI fragment also from pSK-VCGFP. Finally the FIV vector was excised from pSK-RVCGFP by digestion with BamHI and SalI and inserted into pBA-9b(−SD)SIN linearized by digestion with BamHI and XhoI, to generate pMCR-GFP.

### Example 12

### Production from Human or Other Cells of Retroviral or Other Vectors with Enhanced Resistance to Human Complement Inactivation

Resistance to complement inactivation was performed as described in Depolo et al. (1999) J. Virol. 73:6708-6714. VSV-G pseudotyped vector based on MLV, FIV or HIV (prepared essentially as described in Example 6) were sensitive to complement inactivation, even when produced in human 293 cells. In contrast, otherwise-matched amphotropic enveloped retroviral vectors of each of these three types are substantially resistant when produced by the same transfection procedure in human cells (FIG. 5). Thus, these amphotropic vectors are 5, 10, 25 fold more resistant to inactivation by human sera, on average, than the equivalent VSV-G pseudotyped vector. This allows for safer and more efficient delivery of vectors (e.g., by any route including topical, intranasal, oral, intravenous, intramuscular, subcutaneous, intracranial, intraperitoneal, intralesional and the like).

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A chimeric murine leukemia virus (MLV)-feline leukemia virus (FIV) vector construct, comprising an MLV vector backbone and an FIV vector construct.

2. The chimeric vector construct of claim 1, wherein the MLV vector backbone comprises, in 5′ to 3′ orientation, an MLV 5′-LTR, an MLV packaging signal, an MLV polypurine tract (PPT) and an MLV 3′-LTR.

3. The chimeric vector construct of claim 2, wherein the FIV vector construct is inserted in a 5′-3′ orientation between the MLV packaging signal and the MLV PPT.

4. The chimeric vector construct of claim 2, wherein the FIV vector is inserted in a 3′-5′ orientation between the MLV packaging signal and the MLV PPT.

5. The chimeric vector construct of claim 2, wherein the FIV vector construct comprises an FIV 5′ LTR, a tRNA binding site, a packaging signal, one or more genes of interest operably linked to a promoter, an origin of second strand DNA synthesis and a 3′ FIV LTR.

6. The chimeric vector construct of claim 5, wherein the promoter is an FIV LTR promoter or an internal promoter element.

7. The chimeric vector construct of claim 5, wherein the U3 region of one or both of the FIV 5′ LTR and FIV 3′ LTR comprises a heterologous promoter.

8. The chimeric vector construct of claim 7, wherein the heterologous promoter is a viral or non-viral promoter.

9. The chimeric vector construct according to claim 7, wherein the heterologous promoter is a tissue-specific promoter.

10. The chimeric vector construct according to claim 5, further comprising a nuclear transport element selected from the group consisting of MPMV, HBV, RSV and lentiviral Rev-responsive-elements.

11. The chimeric vector construct according to claim 5, wherein the gene of interest is a selectable marker.

12. The chimeric vector construct according to claim 5, wherein said gene of interest is selected from the group consisting of cytokines, factor VIII, factor IX, LDL receptor, prodrug activating enzymes, trans-dominant negative viral or cancer-associated proteins and tyrosine hydroxylase.

13. The chimeric vector construct according to claim 5, wherein the FIV vector further comprises an internal ribosome entry site.

14. The chimeric vector construct according to claim 5, wherein said promoter is operably linked to two genes of interest which are separated by less than 120 nucleotides.

15. A host cell comprising a chimeric vector construct according to claim 1.

16. A method of generating an FIV gene delivery vector comprising:
(a) introducing into a suitable host cell a chimeric vector construct according to claim 1 and one or more elements required for packaging MLV-FIV virions; and
(b) introducing the MLV-FIV virions of step (a) into an FIV packaging cell line, thereby generating an FIV gene delivery vector.

17. The method of claim 16, wherein the one or more elements required for packaging MLV-FIV virions comprise a packaging expression cassette and an envelope expression cassette.

18. The method of claim 17, wherein the packaging expression cassette encodes gag/pol and, optionally, rev, vif or ORF2.

19. The method of claim 17, wherein the envelope expression cassette encodes VSV-G envelope or amphotropic envelope.

20. The method of claim 16, further comprising the step of concentration the MLV-FIV virions prior to step (b).

21. The method of claim 16, wherein the chimeric vector construct and the one or more elements required for packaging MLV-FIV virions are transiently transfected into the host cell.

22. The method of claim 16, wherein the FIV packaging cell line specifically recognizes the packaging signal in the FIV vector.

23. The method of claim 16, wherein the FIV packaging cell line comprises a first expression cassette comprising a promoter operably linked to a sequence encoding gag/pol, a second expression cassette comprising a promoter operably linked to a sequence encoding an envelope, and a nuclear transport element, wherein said promoter is operably linked to said sequence encoding gag/pol.

24. The method of claim 23, wherein the packaging cell line further comprises a sequence encoding one or more of vif rev or ORF 2.

25. The method of claim 23, wherein one or both of said first and second expression cassettes are stably integrated into a cell.

26. The method of claim 23, wherein the sequence encoding gag/pol is derived from FIV and the sequence encoding an envelope is derived from VSV-G or amphotropic envelope.

27. The method of claim 16, wherein the FIV gene delivery vehicles are produced at a concentration of greater than 10³ cfu/ml.

28. The method of claim 16, wherein the FIV gene delivery vehicles are free of replication competent virus.

29. The method of claim 16, wherein said FIV packaging cell line is of feline or human origin.

30. An FIV gene delivery vector produced according to the method of claim 15.
